(19) United States
(12) Patent Application Publication
(10) Pub. No.: US 2002/0094530 A1
(43) Pub. Date: Jul. 18, 2002

(54) Method to identify antibody targets

(76) Inventors: Charles A. Nicolette, Framingham, MA (US); Bruce L. Roberts, Southborough, MA (US)

Correspondence Address: GENZYME CORPORATION C/O MCCUTCHEN, DOYLE, BROWN,, & ENERSEN, MCCUTCHEN, DOYLE, BROWN & ENERSEN, LLP, THREE EMBARCADERO CENTER, SAN FRANCISCO, CA, 94111, US

(21) Appl. No.: 09/955,656

(22) Filed: Sep. 18, 2001

Related U.S. Application Data
(60) Provisional application No. 60/233,586, filed on Sep. 18, 2000.
(60) Provisional application No. 60/262,835, filed on Jan. 19, 2001.
(60) Provisional application No. 60/303,751, filed on Jul. 6, 2001.

(51) Int. Cl. C12Q 1/68; G01N 33/53; G06F 19/00; G01N 33/48; G01N 33/50

(52) U.S. Cl. 435/6; 435/7.1; 702/20; 702/19

(57) ABSTRACT

The present invention provides methods for methods of identifying novel therapeutic polypeptide antigens and epitopes. These methods are designed to select polypeptides that are particularly effective targets for antibody based immunotherapies.

The invention further provides therapeutic polypeptide antigens and epitopes polypeptides that are useful for inducing an immune response in a subject. In addition, the invention provides antibodies directed against these polypeptide antigens and epitopes and methods for using these antibodies to inhibit the progression of disease in a subject.

### CROSS-REFERENCE TO RELATED APPLICATIONS

This application claims priority under 35 U.S.C. § 119 (e) to U.S. Provisional Application Serial Nos. 60/233,586; 60/262,835; and 60/303,751, filed Sep. 18, 2000; Jan. 19, 2001; and Jul. 6, 2001, respectively. The contents of these applications are hereby incorporated by reference into the present disclosure.

### TECHNICAL FIELD

This invention relates to the fields of immunology and antibody technology.

### BACKGROUND OF THE INVENTION

The manipulation of antibody molecules and humoral immune responses directed against normal or mutated cellular antigens expressed in cancers or virally infected cells provides a useful approach for the development of therapeutic and diagnostic agents. Modulation of immune responses may further provide useful strategies for addressing autoimmune diseases and other pathogenic phenotypes. The ability to identify well qualified therapeutic antibody targets will facilitate the diagnosis and treatment of additional diseases.

Antibody-based technologies can be applied in a variety of alternative ways. Vaccine strategies to induce production of antibodies directed against tumor specific antigens can be employed for immunotherapy to produce antibody-dependent cellular cytotoxicity, complement-dependent cytolysis, and apoptosis (Sinkovics and Horvath (2000) Int. J. Oncol. 16(1):81-96). Monoclonal antibodies directed against cell surface tumor antigens have been shown to be directly effective at treating a number of kinds of cancer (Weiner (1999) Semin. Oncol. 26:43-51). Alternatively, antibody immuno-conjugates derived from tumor antigen specific monoclonals are also useful as delivery agents for cytotoxic agents and radionuclides or as imaging agents for diagnostic applications (Roselli et al. (1996) Anticancer Res. 16(4B):2187-2192; Trail and Bianchi (1999) 11(5):584-588). In addition, anti-tumor antibodies have been shown to induce anti-idiotype antibodies that mimic the characteristics of tumor antigens. These anti-idiotype antibodies are capable of further inducing anti-tumor immune responses against the tumor (Fagerberg et al. (1995) 92(11):4773-4777).

Experimental vaccination against cancer also demonstrates that induction of a humoral immune response to tumor antigens is not sufficient to provide a therapeutic effect against the tumor (Sinkovics and Horvath (2000) Int. J. Oncol. 16(1):81-96). Individuals treated with tumor-derived vaccines may or may not respond to the therapy even though both responsive and non-responsive subjects can produce polyclonal antibodies that react with tumor antigens. Identification of the antigenic proteins that induce a positive therapeutic effect in responsive subjects treated would be particularly useful in developing effective immunotherapies.

Viral infections are ideal candidates for immunotherapy. Immunological responses to viral pathogens are sometimes ineffective as in the case of the lentiviruses such as HIV which causes AIDS. The high rates of spontaneous mutation make these viruses elusive to the immune system. However, a saturating profile of CTL epitopes presented on infected cells will identify shared antigens among different serotypes in essential genes that are largely intolerant to mutation which would allow the design of more effective vaccines.

Immune responses to non-infectious antigens can result in pathological states such as, for example, autoimmunity, graft rejection and allergy. Common among such disorders is that the normal mechanisms of the immune response are employed to produce symptoms and pathology and, therefore, significant challenges to treatment are presented. Antibodies and/or antigenic peptides, capable of blocking immune response to self-antigen or diverting the immune response to non-pathogenic pathways, for example, are believed to provide a treatment of such disorders without adversely affecting general immune competence.

Historically, antigenic polypeptides present in pathological cells have been identified by comparing the levels of expression of protein samples derived from pathological and normal cell samples to detect individual antigens that are specifically expressed in the pathological cells. This approach has limited utility in identifying candidates for antibody-based immunotherapy, however, since expression levels are not an indication of cellular localization, accessibility or functional relevance. More recently, the combination of expression cloning and immuno-detection methods has been applied to the identification of tumor derived B-cell antigens. Using the SEREX method (serological identification of antigens by recombinant expression cloning) such antigens have been obtained from a variety of tumor types (WO 00/20460; Tureci et al. (1999) Hybridoma 18(1):23-28). While this method has been useful in finding antigens that elicit a humoral immune response in subjects, this method does not provide “well qualified” antigen targets, because any useful information about the relevance of the identified antigen to disease or its potential utility as a target for immunotherapy is lacking. In fact, many of the antibodies directed against human tumor antigens do not produce a therapeutic effect.

Elucidation of appropriate antigenic polypeptides has been technically difficult. Selection of effective polypeptide targets for antibody technologies requires the identification of accessible antigens with sufficiently restrictive tissue expression patterns to focus the antibody response on the appropriate tissue. For antibody targeting procedures, the abundance and accessibility of the antigenic polypeptide is important, while for therapeutic monoclonal antibody technology, the functional activity of the antigen is critical. Thus, it would be useful to have a means of screening a variety of target pathological cell or tissue samples to identify multiple antigenic polypeptides and antibodies directed against these targets.

This invention satisfies these needs and provides related advantages as well.

### DISCLOSURE OF THE INVENTION

This invention provides a method to identify a polypeptide correlating with a phenotype of interest, wherein the polypeptide specifically recognizes and binds a serum antibody, said method comprising identifying a polypeptide common to a first list of characterized genes, wherein said genes are differentially expressed in one or more relevant cells or tissues and a second list of characterized polypeptides, thereby identify said polypeptide correlating with said phenotype of interest. The polypeptide can be isolated from the serum or can be a membrane-associated polypeptide.

In one aspect, the genes are characterized by cell or tissue type. For example, the genes may all be isolated from cells or tissue suspected of expressing the antigen of interest, a cancer cell, a normal cell, a virally infected cell or a cell associated with an autoimmune disorder.

In a further aspect, the genes of the first list are characterized by properties of the gene product, wherein said properties are selected from the group consisting of specific reactivity with the serum antibody, highly expressed in the relevant cell line or tissue, little or no detectable expression in the relevant cell line or tissue, and uniquely expressed in the relevant cell line or tissue. In a yet further aspect, the list is generated or common to cells or tissues having two or more of these properties. In a still a further embodiment, all cells and tissues are selected due to an expression product having a molecular weight within a certain range, i.e., a range similar to or which encompasses the molecular weight of the antigen or polypeptide which binds to the serum antibody.

In another embodiment, the second list is defined by common characteristics of the proteins, wherein such characteristics or properties are selected from the group consisting of mass, reactivity with the serum antibody, peptidase digestion pattern, enzymatic digestion pattern and MOLDI-TOF (Matrix Assisted Laser Desorption Ionization Time of Flight Mass Spectrometry) selection criteria. In a yet further aspect, the list is generated or common to cells or tissues having two or more of these properties. In a still a further embodiment, all cells and tissues are selected due to an expression product having a molecular weight within a certain range, i.e., a range similar to or which encompasses the molecular weight of the antigen or polypeptide which binds to the serum antibody.

MOLDI-TOF is a method to identify proteins based on mass spectrometry data. Examples of MOLDI-TOF selection criteria include but are not limited to database selection, species, type of digest, CNBr, number of miscleavages, molecular weight range, contamination indication, and mass accuracy.

The present invention provides also provides methods to identify a serum protein, polypeptide or epitope correlating with a phenotype of interest, for example cancer, autoimmune disease, viral pathogens, and including degenerative disorders such as polycystic kidney disease.

In one embodiment, serum protein is isolated from one or more “responsive subjects” i.e., one or more which possesses a desired phenotype, e.g., the presence of a clinical or subclinical response. A subject can exhibit a positive response to an intervention such as reduction of tumor burden in the case of cancer, a reduction in the number of autoimmune antigens or antibodies in the case of autoimmune disease such as systemic lupus erythematosus and a reduction in bone de-mineralization in the case of polycystic kidney disease. However, the invention is not limited to cases of response to therapeutic intervention. Responsive subjects also include those who have been exposed to pathogens or alternatively, having genetic predisposition to disease. In each case, “responsive” subjects include those who do not exhibit clinical or subclinical symptoms associated with the correlative phenotype, e.g., AIDS after exposure to and/or the presence of HIV in the subject or infection after exposure to pathogen. Serum is isolated from one or more “non-responsive” subjects, i.e., those exhibiting the pathogenic phenotype such as cancer, infection or HIV-associated disease such as AIDS. A serum antibody which is differentially present or absent between the responsive serum and the non-responsive serum is identified by assay of the serum against a panel of polypeptides.

In yet a different embodiment, serum is isolated from more than one non-responsive subjects. Serum antibodies which are shared by at least two or more of the subjects may be identified by assay of the serum against a panel of polypeptides. The antibodies are recovered and screened for those that are differentially present or absent by comparison to expressed genetic or peptide data for the phenotype of interest.

In a further embodiment, once antibodies are recovered, they are screened for those that are differentially present or absent are isolated and further analyzed by comparison to expressed genetic data for the phenotype of interest. In a preferred embodiment, the expressed genetic data will be selected to share some properties with the test subjects. In a most preferred embodiment, the expressed genetic data will be from tissues or cells matched to share as many properties as possible. A particularly preferred embodiment is the comparison of the genetic profile of from a responder with a non-responder.

Alternatively, the antibodies are recovered and screened by chromatographic and genetic assays to identify the polypeptide and its polynucleotide sequence.

The invention further provides the polypeptide isolated by the method and methods to confirm diagnostic and therapeutic utility. Yet further provided are antibodies (monoclonal and polyclonal) that specifically recognize and bind the polypeptide(s). Also provided are polynucleotides encoding the polypeptides and antibodies.

Still further provided are isolated cells and cell-surface ligands that specifically bind a polypeptide or antibody of this invention. Still further provided are derivatives of each of the polypeptide, antibody, cell, and cell-surface ligand, e.g., a labeled polypeptide for use in a diagnostic kit or as a drug molecule carrier, a humanized antibody for immunotherapy or antibody fragment for use as a drug molecule carrier, an antigen-presenting cell containing a polynucleotide encoding a polypeptide of this invention and soluble ligand for use to block binding of an antibody or antigen to its target, respectively. Reagents and kits to perform these methods are further provided as well as use of the antibodies and antigens possessing therapeutic and/or diagnostic utility.

Another aspect of the invention is a method for identification of a ligand which binds an antibody, the presence or absence of which has been correlated with a phenotype in a subject. A sample of cells suspected of containing ligand is isolated from a subject and contacted with an effective amount of an antibody or its derivative which has been detectably labeled. The ligand is identified by its binding to the antibody or derivative. Methods to isolate the ligand from the cells containing the ligand are known in the art. Soluble ligand also can be isolated by removal of the cytoplasmic and transmembrane domain of the ligand. Polynucleotides encoding the ligand and its soluble form are further provided herein as well as compositions containing one or more of an isolated ligand, a polynucleotide encoding the ligand, host cells containing and/or the ligand or polynucleotide encoding the ligand are further provided herein.

A test compound which diminishes the binding of the polypeptide or antibody to its partner is further provided herein as well as methods for its identification. A screen to identify these molecules is performed by contacting a sample of polypeptide or antibody with its binding partner in the presence and absence of the test compound. A molecule that inhibits the binding between antibody and polypeptide or ligand as compared to the sample which does not include the test compound (the control sample) is identified as a molecule which diminish the binding of antibody to polypeptide or ligand.

A still further embodiment of the invention is a method to identify candidate drugs for treating tumors or other pathologies identified herein. Cells which express one or more polynucleotides of the invention are contacted with a test compound. Expression of the one or more polynucleotides is determined by hybridization of mRNA of the cells to a nucleic acid probe which is complementary to said mRNA. A test compound is identified as a candidate drug for treating tumors if it decreases expression of the one or more polypeptides of this invention. Alternatively or additionally, the cells are recombinant host cells which are transfected with an expression construct which encodes one or more polypeptides.

### BRIEF DESCRIPTION OF THE DRAWINGS

FIG. 1 shows that antibody eluted by the protocol provided herein retains its specific antigen binding property. The blot on the right was generated with anti-Her2 antibody eluted from the blot on the left.

FIG. 2 is a schematic of the Tracer or Indirect Method using human anti-Her2 antibodies.

FIG. 3 shows a Western blot from which the Her-2 antibody was eluted (top) and one of the “positive” beads that was sequenced (middle) using the method described herein. The bottom of the Figure shows an alignment of the two library sequences with a region of the Her-2 protein.

FIG. 4 shows a schematic for isolation of apical and basolateral cell membranes.

FIG. 5 is a schematic of an alternate embodiment of the invention that utilizes MALDI-TOF analysis to identify the antigen of interest.

FIG. 6 shows the band of antibodies isolated from a responsive melanoma patient immunized with a MART-1 vaccine. Pre- and post-vaccination serum are shown.

FIG. 7 is a Western blot confirmatory analysis of immunopreciptitated polypeptide (protein) isolated by the method of this invention. Using conventional ELISA, the polypeptide is shown to specifically recognize and bind IgM antibodies isolated from the responsive patient serum. The band is noticeably diminished in the center lane (IP supernatant).

FIG. 8 is a Western blot of a commercially available monoclonal antibody bound to isolated peptide (protein) identified in FIG. 7.

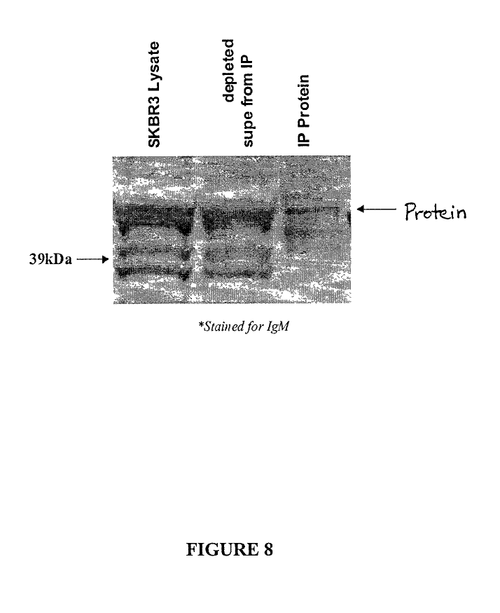

### MODES OF CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. These methods are described in the following publications. See, e.g., Sambrook et al. MOLECULAR CLONING: A LABORATORY MANUAL, 2^{nd} edition (1989); CURRENT PROTOCOLS IN MOLECULAR BIOLOGY (F.M. Ausubel et al. eds. (1987)); the series METHODS IN ENZYMOLOGY (Academic Press, Inc.); PCR: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1988)) USING ANTIBODIES, A LABORATORY MANUAL (Harlow and Lane eds. (1999)); and ANIMAL CELL CULTURE (R. I. Freshney ed. (1987)).

Definitions

As used herein, certain terms may have the following defined meanings.

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “native” or “natural” antigen is a polypeptide, protein or a fragment thereof which contains an epitope, which has been isolated from a natural biological source, and which can specifically bind to an antigen receptor in a subject, for example, T cell antigen receptor (TCR), B cell antigen receptor (BCR) or surface immunoglobulin.

The term “antigen” is well understood in the art and includes substances which induce a response in the subject. The response may be subclinical, e.g., reduction in viral load in a subject or clinical such as reduction in tumor burden in the case of cancer.

An “altered antigen” is one having a primary sequence that is different from that of the corresponding wild-type antigen. Altered antigens can be made by synthetic or recombinant methods and include, but are not limited to, antigenic peptides that are differentially modified during or after translation, e.g., by phosphorylation, glycosylation, cross-linking, acylation, proteolytic cleavage, linkage to an antibody molecule, membrane molecule or other ligand (Ferguson et al. (1988) Ann. Rev. Biochem. 57:285-320). A synthetic or altered antigen of the invention is intended to bind to and/or cross-react with the same TCR as the natural epitope.

A “self-antigen” also referred to herein as a native or wild-type antigen is an antigenic peptide that induces little or no immune response in the subject due to self-tolerance to the antigen. An example of a self-antigen is the melanoma specific antigen gp100.

The term “tumor associated antigen” or “TAA” refers to an antigen that is associated with or specific to a tumor. Examples of known TAAs include gp100, MART and MAGE.

“Responsive subject serum (antiserum)” refers to a polyclonal serum sample obtained from a subject showing a desired response after a treatment as compared with a responsive polyclonal serum sample isolated or derived from a similar subject, e.g., receiving the same treatment but does not exhibit desired response, i.e., a “non-responsive subject serum”. In one aspect, the serum from a responsive subject will be obtained from a subject exhibiting a clinical or subclinical response.

The term “antigen presenting cells (APCs)” refers to a class of cells capable of presenting one or more antigens in the form of antigen-MHC complex recognizable by specific effector cells of the immune system and, thereby, inducing an effective cellular immune response against the antigen or antigens being presented. While many types of cells may be capable of presenting antigens on their cell surface for T cell recognition, only professional APCs have the capacity to present antigens in an efficient amount and further to activate T cells for cytotoxic T-lymphocyte (CTL) responses. APCs can be intact whole cells such as macrophages, B cells and dendritic cells (DCs); or other molecules, naturally occurring or synthetic, such as purified MHC class I molecules complexed to β2-microglobulin.

The term “dendritic cells (DCs)” refers to a diverse population of morphologically similar cell types found in a variety of lymphoid and non-lymphoid tissues (Steinman (1991) Ann. Rev. Immunol. 9:271-296). Dendritic cells constitute the most potent and preferred APCs in the organism. A subset of, if not all, dendritic cells are derived from bone marrow progenitor cells, circulate in small numbers in the peripheral blood and appear either as immature Langerhans' cells or terminally differentiated mature cells. While the dendritic cells can be differentiated from monocytes, they possess distinct phenotypes. For example, a particular differentiating marker, CD14 antigen, is not found in dendritic cells but is possessed by monocytes. Also, mature dendritic cells are not typically phagocytic, whereas monocytes are strongly phagocytosing cells. It has been shown that DCs provide all the signals necessary for T cell activation and proliferation.

The term “immune effector cells” refers to cells capable of binding an antigen and which mediate an immune response. These cells include, but are not limited to, T cells, B cells, monocytes, macrophages, NK cells and cytotoxic T lymphocytes (CTLs), for example, CTL lines, CTL clones, and CTLs from tumor, inflammatory, or other infiltrates. Certain diseased tissues express specific antigens and CTLs specific for these antigens have been identified. For example, approximately 80% of melanomas express the antigen known as gp100.

The term “immune effector molecule” as used herein, refers to molecules capable of antigen-specific binding, and includes antibodies, T cell antigen receptors, and MHC class I and class II molecules.

A “naïve” immune effector cell is an immune effector cell that has never been exposed to an antigen.

As used herein, the term “educated, antigen-specific immune effector cell”, is an immune effector cell as defined above, which has encountered antigen and which is specific for that antigen. An educated, antigen-specific immune effector cell may be activated upon binding antigen. “Activated” implies that the cell is no longer in G₀ phase, and begins to produce cytokines characteristic of the cell type. For example, activated CD4⁺ T cells secrete IL-2 and have an increased number of high affinity IL-2 receptors on their cell surfaces relative to resting CD4⁺ T cells.

A peptide or polypeptide of the invention may be preferentially recognized by antibodies or/and antigen-specific immune effector cells, such as B cells and T cells. In the context of T cells, the term “recognized” intends that a peptide or polypeptide is processed and presented on the surface of an APC together with (i.e., bound to) an MHC molecule in such a way that a T cell antigen receptor (TCR) on the surface of an antigen-specific T cell binds to the epitope wherein such binding results in activation of the T cell. In the context of B cells, upon binding of an antigen to the B cell receptor (BCR) the antigen is internalized and returned to the cell surface as peptides bound to MHC class II molecules. This peptide:MHC complex is “recognized” by antigen-specific T cells which then activate B cells to secrete antibodies. The term “preferentially recognized” intends that a polypeptide of the invention is substantially not recognized, as defined above, by a T cell specific and/or antigen for an unrelated antigen. Assays for determining whether an epitope is recognized by an antibody or antigen-specific T cell are known in the art and are described herein. In the context of antibody:antigen binding, “specifically recognized and binds” means that the members of the pair are substantially not recognized by other binding partners.

As used herein, “solid phase support” or “solid support”, used interchangeably, is not limited to a specific type of support. Rather, a large number of supports are available and are known to one of ordinary skill in the art. Solid phase supports include silica gels, resins, derivatized plastic films, glass beads, cotton, plastic beads, alumina gels. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc. (King of Prussia, Pa.), Peninsula Laboratories Inc. (San Carlos, Calif.), etc.), POLYHIPE® resin (obtained from Aminotech, Canada), polyamide resin (obtained from Peninsula Laboratories Inc.), polystyrene resin grafted with polyethylene glycol (TentaGel®, Rapp Polymere, Tubingen, Germany) or polydimethylacrylamide resin (obtained from Milligen/Biosearch, Novato, Calif.).

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-stranded, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

The term “genetically modified” means containing and/or expressing a foreign gene or nucleic acid sequence which in turn, modifies the genotype or phenotype of the cell or its progeny. In other words, it refers to any addition, deletion or disruption to a cell's endogenous nucleotides.

As used herein, “expression” refers to the process by which polynucleotides are transcribed into MRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook et al. (1989) supra). Similarly, an eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

A “gene or polynucleotide library” is a library of nucleotide sequences isolated from cells or tissues suspected of containing the gene encoding the antigen (polypeptide) of interest.

A “peptide library” is a combinatorial library of random amino acid (peptide) sequence.

The term “sequence motif” refers to a pattern present in a group of molecules (e.g., amino acids or nucleotides). For instance, in one embodiment, the present invention provides for identification of a sequence motif among peptides present in an antigen. In this embodiment, a typical pattern may be identified by characteristic amino acid residues, such as hydrophobic, hydrophilic, basic, acidic, and the like.

“Under transcriptional control” is a term well understood in the art and indicates that transcription of a polynucleotide sequence, usually a DNA sequence, depends on its being operably (operatively) linked to an element which contributes to the initiation of, or promotes, transcription. “Operably linked” refers to a juxtaposition wherein the elements are in an arrangement allowing them to function.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6× SSC to about 10× SSC; formamide concentrations of about 0% to about 25%; and wash solutions of about 6× SSC. Examples of moderate hybridization conditions include: incubation temperatures of about 40° C. to about 50° C.; buffer concentrations of about 9× SSC to about 2× SSC; formamide concentrations of about 30% to about 50%; and wash solutions of about 5× SSC to about 2× SSC. Examples of high stringency conditions include: incubation temperatures of about 55° C. to about 68° C.; buffer concentrations of about 1× SSC to about 0.1× SSC; formamide concentrations of about 55% to about 75%; and wash solutions of about 1× SSC, 0.1× SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The terms “major histocompatibility complex” or “MHC” refers to a complex of genes encoding cell-surface molecules that are required for antigen presentation to T cells and for rapid graft rejection. In humans, the MHC complex is also known as the HLA complex. The proteins encoded by the MHC complex are known as “MHC molecules” and are organized into class I and class II MHC molecules. Class I MHC include membrane heterodimeric proteins made up of an α chain encoded in the MHC noncovalently linked with the β2-microglobulin. Class I MHC molecules are expressed by nearly all nucleated cells and have been shown to function in antigen presentation to CD8+ T cells. Class I molecules include HLA-A, B, and C in humans. Class II MHC molecules also include membrane heterodimeric proteins consisting of noncovalently associated α and β chains. Class II MHC molecules are known to function in CD4+ T cells and, in humans, include HLA-DP, -DQ, and DR.

The term “autologous” or “autogeneic”, as used herein, indicates the origin of a cell. The term indicates that cell samples are derived from the same subject or from a donor genetically identical to the subject. An autologous cell can also be a progeny of an autologous cell The term also indicates that cells of different cell types are derived from the same subject or from donors genetically identical to the subject.

Similarly, the term “allogeneic” indicates the origin of a cell. The term indicates that cell samples are derived from donors not genetically identical to the subject; in particular, the term relates to non-identity in expressed MHC molecules. An allogeneic cell can also be a progeny of an allogeneic cell. The term also indicates that cells of different cell types are derived from donors not genetically identical to the subject.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors and the like. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., WO 95/27071. Ads are easy to grow and do not require integration into the host cell genome. Recombinant Ad-derived vectors, particularly those that reduce the potential for recombination and generation of wild-type virus, have also been constructed. See, WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., TCR, CD3 or CD4.

A polynucleotide or polynucleotide region (or a polypeptide or polypeptide region) has a certain percentage (for example, 80%, 85%, 90%, or 95%) of “sequence identity or homology” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: www.ncbi.n1m.nih.gov/cgi-bin/BLAST.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced to a cell of such organism in vivo.

The term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

A “subject” is a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative”. For example, where the purpose of the experiment is to determine a correlation of an altered expression level of a gene with a particular type of cancer, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

“Immunization” or “vaccination” shall mean increasing or activating an immune response against an antigen. It does not require elimination or eradication of a condition but, rather, contemplates the clinically favorable enhancement of an immune response toward an antigen.

“Suppressing” tumor growth or “reducing tumor burden” in a subject indicates a growth state that is curtailed compared to a control. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth means any or all of the following states: slowing, delaying, and “suppressing” tumor growth indicates a growth state that is curtailed when stopping tumor growth, as well as tumor shrinkage.

The term “culturing” refers to the in vitro propagation of cells or organisms on or in media of various kinds. It is understood that the descendants of a cell grown in culture may not be completely identical (morphologically, genetically, or phenotypically) to the parent cell. By “expanded” is meant any proliferation or division of cells.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin REMINGTON'S PHARM. SCI., 15th Ed. (Mack Publ. Co., Easton (1975)).

An “effective amount” is an amount sufficient to effect beneficial or desired results. An effective amount can be administered in one or more administrations, applications or dosages.

“Serum” sample refers to the fluid phase of blood or plasma (collected from a subject) that contains circulating antibodies and other soluble proteins. In a preferred aspect, a serum sample contains antibodies or proteins that correlate with a phenotype of interest. In one aspect, a “phenotype of interest” is a particular disease state. In another aspect, a “phenotype of interest” is characterized by the presence of antibodies or proteins that correlate with an immune response to a particular treatment.

“Responsive” subject serum (antiserum)” refers to a polyclonal serum sample obtained from a subject showing a desired response after a treatment as compared with a a polyclonal serum sample isolated or derived from a similar subject, e.g., receiving the same treatment but does not exhibit a desired response, i.e., a “non-responsive subject serum”. In one aspect, the serum from a responsive subject will be obtained from a subject exhibiting a clinical or subclinical response.

Antigenic polypeptides and antibodies that specifically recognize and bind to polypeptides are potentially useful as therapeutic agents. However, only a subset of the antigenic polypeptides expressed by antigen-containing or potential antigen-containing cells are effective targets for immuno- or drug therapy. Current methods for identifying antigenic peptides and associated antibodies do not select antigens on the basis of their potential utility. The present invention provides a means to select antigenic polypeptides the presence or absence of which are associated with a desired response or phenotype by providing a method to identify a polypeptide correlating with a phenotype of interest, wherein the polypeptide specifically recognizes and binds a serum antibody, said method comprising identifying a polypeptide common to a list of characterized genes, wherein said genes are differentially expressed in one or more relevant cells or tissues and a list of characterized polypeptides, thereby identify said polypeptide correlating with said phenotype of interest. The serum antibody is selected from two or more phenotypes of interest. In one aspect, this is accomplished by selecting serum from subjects that are compared according to a predetermined phenotype, i.e., they express a differential in a phenotype common to each, e.g., differential response to treatment with a cancer vaccine. Characterized gene expression data is then obtained and a list is obtained through the identification and application of characteristics common the antigen or polypeptide and the list. For example, if the serum antibody is selected from a patient having been treated with cancer, the antigen may first be characterized by its ability to bind to the antibody, (e.g., by Western blot) wherein the antigen is selected from cell or tissue types suspected of expressing the antigen of interest and for which gene expression data is available and has been characterized. This may also provide the molecular weight of the antigen. The database of expressed gene sequences can be further selected or limited by additional common characteristics common to the antigen of interest, methods for determining such characteristics are provide herein.

The second list is generated from separate but relevant protein data, e.g., molecular weight, enzymatic digestion pattern and the light. These properties are common to the antigenic peptide, the properties of which can be determined after isolation of additional antigen from the appropriate sample and using the methods provided herein.

The lists are compared and based on the separate lists relating to related or common phenotypic information, at least one polypeptide common to each list is identified. Based on the selection criteria, this polypeptide binds to a serum antibody isolated from a subject having a pre-selected phenotype.

Selection of the Polyclonal Samples Produced In Vivo or In Vitro

In one aspect, the desired response is observed after a treatment. In another aspect, a desired response is observed in a subject in the absence of a treatment.

As used herein, a “treatment” or a “therapy” intends any intervention that increases or activates a response in the host or subject. In some aspects, a therapeutic response is a clinical response, although sub-clinical responses are within the scope of this invention as well. Treatments that may lead to a response include, but are not limited to antibiotic therapy, anti-viral therapy, vaccination, immunization, drug therapy (e.g., small molecules) chemotherapy, radiation therapy, successful resolution of disease after primary surgery, antibody therapy, passive immune therapy, active immune therapy, adoptive immune therapy and the like.

The therapeutic response will vary with the subject being treated and the object of the treatment. Examples include humoral and cellular immune responses, the generation of anti-antigen antibodies, generation of cytotoxic T cells specific for the antigen which will lyse cells displaying the antigen, a non-specific innate immune response, e.g., activation of NK cells, phagocytes or macrophages. Methods for determining whether an immune response has been induced are well known in the art. For example, antigen-specific antibody can be detected using any of a variety of immunoassays known in the art, including, but not limited to, ELISA, wherein, for example, binding of an antibody to an immobilized antigen (or epitope) is detected with a detectably-labeled second antibody (e.g., enzyme-labeled mouse anti-human Ig antibody). Immune effector cells specific for the antigen can be detected in any of a variety of assays known to those skilled in the art, including, but not limited to, FACS, ⁵¹Cr-release assays, or ³H-thymidine uptake assays.

In one aspect of this invention, serum samples containing polyclonal antibodies are isolated from two or more responsive subjects. In one aspect, a sample containing antibodies are isolated from two or more subjects, who are subsequently treated. Post-treatment serum samples are isolated from two or more subjects. When the subjects' respective immune responses are phenotypically distinguishable, e.g., from both a subject who was “responsive” to the given treatment and a subject who was “non-responsive” to the given treatment. In this example the desired response produced by the responsive subject is a therapeutic immune response.

In yet a different aspect of this invention, phenotypically distinct serum samples for analysis and identification of the therapeutic antigen and antibody can be derived from in vivo or in vitro screening of subject samples. Examples are provided herein.

The subject method can also be practice with samples obtained in vitro using a combinatorial antibody library (antibody phage) which has been panned on tumor cells, tumor cell membranes or shed antigens from tumor cells in vitro to derive a polyclonal population of binding antibodies. The polyclonal antibodies are absorbed using the above-described methods to normal cells to eliminate pan reactive antibodies that recognize ubiquitous antigens and then seek to demonstrate either in vitro or in vivo that the population of enriched antibodies modulates the behavior of target cells.

Selection and Preparation of Target Peptides

In order to identify the serum antibody and its polypeptide target antigen, protein (polypeptide) preparations from target sample/cells suspected of containing the antigen or polypeptide are isolated from whole cell or tissue lysates. Alternatively, since many relevant antibody targets are naturally present on the cell surface, preparations of plasma membranes (e.g., membrane proteins) which are membranes are highly enriched for cell surface proteins can be used. Still further, selective preparations of only apical and basolateral cell membrane proteins are used.

Proteins within whole cell lysates, membrane preparations or enriched preparations of the target samples are arrayed or separated, for example, by molecular weight via electrophoresing, on a protein gel such as an SDS polyacrylamide gel. Identical arrays of the target samples are prepared, one for each serum sample to be assayed. Once separated the proteins are then transferred to a suitable assay medium, e.g., solid support, such as nitrocellulose, bead, and the like, or a liquid carrier. Techniques for manipulating proteins are well known to those skilled in the art. Specific details of such techniques can be obtained from well known sources such as CURRENT PROTOCOLS IN MOLECULAR BIOLOGY (F. M. Ausubel et al. eds. (1987)); and the series METHODS IN ENZYMOLOGY (Academic Press, Inc.).

Isolation of the Serum Polypeptide

In one aspect of the invention, each serum sample is then individually and independently assayed against separate arrays of target sample prepared as described, infra using standard immunological assays for detecting and/or measuring antibody-antigen binding. Western blot analysis is a well known example of such methods.

The serum antibody is then rescued or eluted from the nitrocellulose without destroying the antibody's functionality using a modification of the method of Maa, J. S. et al. (1990) J. Biol. Chem. 265:1569-1577. However, any method that removes antibody without loss of functionality can be used.

Enrichment of Serum Peptide

Isolation of additional antigenic peptide may be necessary for identification of the antigen. Additional antigen can be isolated using immunochemical techniques. In one aspect of the present invention, the antibody is used to immunoprecipitate the antigen from the expressing target sample/cell line. Those of skill in the art are familiar with a variety of techniques that may be employed to identify a protein of interest. For example, methods for immunoprecipitating are described in Harlow and Lane eds. (1988) and (1999) supra or by the direct and/or indirect method described infra.

Identification Screens

The antigen can be identified using a variety of techniques selected from the group consisting of: 1) direct sequencing, 2) direct sequencing and comparison with a gene or polynucleotide database, 3) amino acid sequencing comparison with a combinatorial peptide library (on and off bead); 4) immunoprecipitation with a peptide library (on and off bead), sequencing and comparison to a gene or polynucleotide database, 5) peptidase digestion, mass spectroscopy and comparison with database of known and previously characterized proteins and 6) mass spectroscopy and comparison with database of known and previously characterized proteins.

Polypeptide Library Screens

In some embodiments and prior to identification, the isolated serum antibody is screened against a second panel or library of peptides selected from a sample suspected of containing the antigen of interest using standard immunochemical techniques. Examples of these methods are known in the art (Sambrook et al., (1989) supra and Harlow and Lane (1988) and (1999) supra) or described infra. These assay can be performed directly on with a blocking antibody as described herein.

Alternatively, a combinatorial library of random peptides can be used. In a further embodiment, the library has been pre-selected to increase the probability that the antigen of interest is present.

“Gene”-Based Identification

The polynucleotide sequence of the isolated peptide can be obtained using conventional sequencing techniques or commercially available materials. The peptide can be obtained subsequent to the second screen against the peptide library. In most instances, the sequence of the peptide is insufficient to provide the identify of the protein containing the epitope. This invention provides the means to identify the protein by comparing the sequence against a database of characterized expressed polynucleotide sequences.

Most suitable, the database(s) used to identify the protein are derived from target samples, target cell lines or cells or tissue expressing the same or similar phenotype. The term “target sample” or “target cell line” intends a biological sample that has gene expression profile data associated with it. For example, the target sample can be a normal tissue sample, an abnormal sample, a tumor sample, or a cell line which has an associated gene expression profile. DNA and Protein databases are commercially available (Incyte Genomics, California USA) publicly available (e.g., http://fasta.bioch.virginia.edu/fasta/, http://www.ncbi.nlm.nih.gov/BLAST/; http://blast.wustl.edu; http:/Hexpasy.ch/ ) or can be derived de novo.

Alternatively, one can use any method known in the art used to identify differentially expressed polynucleotides and each can be used in invention methods. As used herein, the term “polynucleotide fragment” includes SAGE tags (described in U.S. Pat. No. 5,695,937) as well as any other nucleic acid obtained from any methods that yield quantitative/comparative gene expression data. Such methods include, but are not limited to, cDNA subtraction, differential display and expressed sequence tag methods. Techniques based on cDNA subtraction or differential display can be quite useful for comparing gene expression differences between two cell types (Hedrick et al. (1984) Nature 308:149; and Lian and Pardee (1992) Science 257:967). The expressed sequence tag (EST) approach is another valuable tool for gene discovery (Adams et al. (1991) Science 252:1651), like Northern blotting, RNase protection, and reverse transcriptase-polymerase chain reaction (RT-PCR) analysis (Alwine et al. (1977) PNAS 74:P5350; Zinn et al. (1983) Cell 34:865; and Veres et al. (1987) Science 237:415). A further method is differential display coupled with real-time PCR and representational difference analysis (Lisitisyn and Wigler (1995) Meth. Enzymol. 254:291).

It should be understood, although not always explicitly stated, that the selection of target samples/cells from which the gene or polynucleotide database as derived and used for sequence comparison will vary depending on the respective indication being treated, e.g., cancer, autoimmune, viral or parasitic. For example, if the indication is viral, the selected target sample/cell is assayed in both an uninfected state and in a state of infection with the virus of interest. Additional samples of the infected target sample/cell may also be included in the assay. For example, target samples that have been infected over a time course representative of the various stages of viral infection, are useful to monitor and detect the temporal components of infection—e.g., immediate early, constant and late gene expression.

When the length of the amino acid sequence that is being compared with the database is small, it is advantageous to have a pre-selected collection of sequenced amino acids sequences to search for the identity of the query sequence in order to simplify the search procedure. Information about the specific set of genes expressed by the target sample and the characteristics of the proteins that react uniquely with antibodies present only in a serum sample can be used to create a pre-selected set of proteins.

Because only a portion of the sequence of a selected antibody reactive peptide may be identical to the sequence of the corresponding natural epitope it is further advantageous to use a pre-selected group of polypeptide sequences when screening for the natural epitope. This increases the efficiency of the screening process by eliminating irrelevant sequences that may share partial homology with the identified antigenic peptides.

Identification of the Serum Peptide With Peptide Databases

In one embodiment of the invention, the amino acid sequence of the serum peptide is determined by comparison to known and previously characterized peptides.

For example, the peptide sequence of the epitope can be determined using methods that include, but are not limited to the “phage method” (Scott and Smith (1990) Science 249:386; Cwirla et al. (1990) PNAS 87:6378; and Devlin et al. (1990) Science 249:404), the Geysen method (Geysen et al. (1986) Mol. inmunol. 23:709: and Geysen et al. (1987) J. Inmunol. Meth. 102:259), the method of Fodor et al. (1991) Science 251:767), methods to test peptides that are agonists or antagonists as described in Furka et al. (1988) 14^{th} International Congress of Biochemistry Vol. 5 Abstract FR:013; Furka (1991) Int. J. Peptide Protein Res. 37:487); Houghton (U.S. Pat. No. 4,631,211); and Rutter et al. (U.S. Pat. No. 5,101,175), the method utilizing synthetic libraries (Needels et al. (1993) PNAS 90:10700; and Lam et al., U.S. Pat. No. 5,510,240), the method that utilizes indexed combinatorial peptide displays (Ohlmeyer et al. (1993) PNAS 90:10922) and the pepscan technique (Van der Zee (1989) Eur. J. Immunol. 19:43).

In embodiments employing peptide library screens, most preferably, peptides of the present invention can be synthesized using an appropriate solid state synthetic procedure; Steward and Young, SOLID PHASE PEPTIDE SYNTHESIS, Freemantle, San Francisco, Calif. (1968). A preferred method is the Merrifield process. See, Merrifield (1967) Recent Progress in Hormone Res. 23:451. The amino acid sequence of the antigenic epitope is then determined and compared with the amino acid sequences encoded by a plurality of genes in a pre-selected set of sequences expressed in the target tissue. Polypeptide sequences encoded by genes in the pre-selected group that share sequence homology with the amino acid sequence of the antibody binding peptide will define a natural epitope recognized and bound by the antibody from the serum.

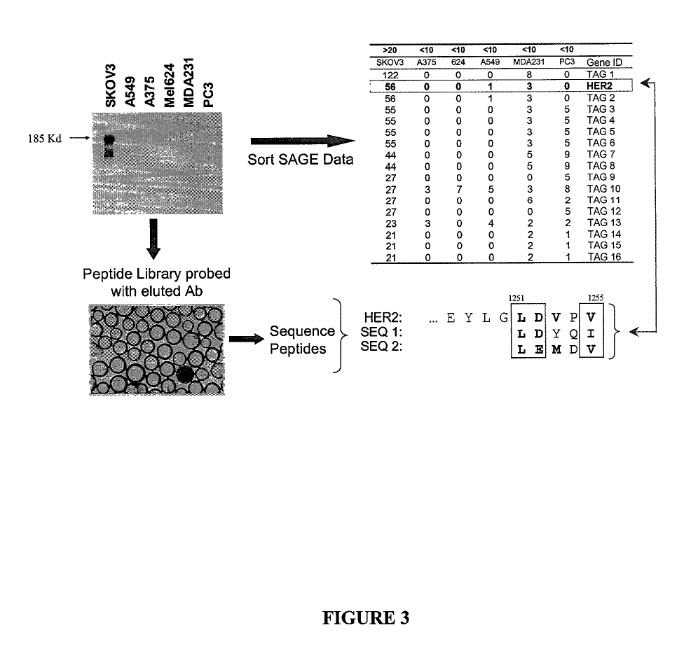

When the antigenic epitope present in a polypeptide that reacts uniquely with an antibody in the serum of a subject is identified using the method described herein, it is advantageous to additionally identify a plurality of antigenic peptides that each react with the selected antibody. Because the binding of an antibody to a polypeptide substrate depends on the three dimensional structure of the substrate, and frequently not all of the residues in the epitope make direct contact with the antibody molecule, more than one specific peptide sequence may be found to react with a selected antibody. Alignment of a plurality of identified antibody binding peptides enables the determination of a consensus sequence (or sequence motif) of the epitope to facilitate identification of the natural epitope in a protein database (See FIG. 3).

MALDI-TOF MS

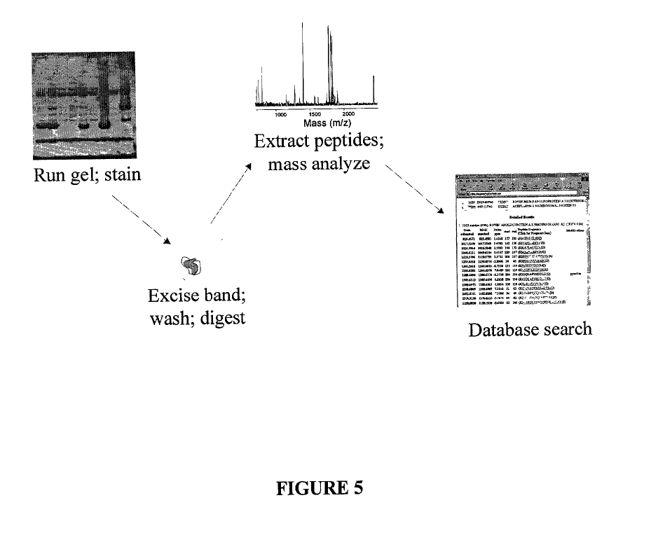

In this embodiment, Matrix Assisted Laser Desorption Ionization Time of Flight Mass Spectrometry (“MALDI-TOF MS”) is used to identify the peptide targets recognized by antibodies shown to possess reactivity (see FIG. 5). In this aspect, after the antibody is isolated or alternatively, the serum in which antibody is present is identified (See FIG. 5), an additional protein gel is run to isolate the peptide. The band is excised and the peptide is isolated and prepared for analysis through the mass spectrometer by digestion with one or more known and previously characterized peptidase. As used herein, the term “known and previously characterized peptidase” intends a protein or peptide for which the fragments of a known peptidase digestion have been previously identified, characterized and cataloged.

For example, using well known procedures described for example in Shevchenko, A et al. (2000) Anal. Chem. 72:2132-2141, the sample or unknown peptide is mixed with matrix and dried on a sample plate. The sample is introduced into the high vacuum of the mass spectrometer. The sample spot is irradiated with laser, desorbing ions into the gas phase and starting the clock measuring the time of flight. Ions are accelerated by an electrical field to the same kinetic energy, and they drift (or fly) down a field free flight tube where they are separated into space. Ions strike the detector at different times depending on the mass to charge ratio of the ion. A data system controls all instrument parameters, acquires the signal versus time, and permits data processing. The spectrometer generates a profile of the sample which is then compared against previously characterized proteins and peptides. Peptide mass spectrometry databases are available on the web, e.g., http://ntwade/htmlucsf/msfit.htm, or can be internally generated. Matches identify the targets recognized by antibodies shown to possess reactivity.

Confirmation of Biological Activity

Polyclonalor monoclonal antibodies and peptides containing the epitope of interest can be prepared and isolated using invention methods.

The functional activity or relevance of the isolated antigen or peptide can be confirmed using well known in vitro or in vivo methods. Therapeutic and diagnostic utility is confirmed by administration of the peptide or antigen to a subject in need of treatment. For example, a target sample/cell line (the gene expression profile of which is known) from a human subject is used to immunize mice to elicit a specific humoral immune response against a polypeptide antigen expressed by the target sample/cell. Means for producing a more robust humoral response in mammals are known in the art. For example, the mice selected to receive the immunization can be naturally T cell deficient or rendered T cell deficient by treatment with anti-CD8 antibody to suppress T cell response.

Methods for demonstrating that the humoral immune response elicited by the immunization also confers protective immunity are known in the art, for example, adoptive transfer of antibodies obtained from the immunized (protected) mice to a group of naïve mice. The naïve mice receiving these antibodies are then challenged with the target sample/cell line and/or other antigenically related cells and monitored for immune responses thereto. Monoclonal and polyclonal antibodies may be obtained from the immunized mice using standard techniques.

These antibodies (monoclonal or polyclonal) can also be assayed in vitro to determine their ability to inhibit proliferation of human tumor cells. The specificity of antibodies shown to inhibit human tumor cell proliferation in vitro can be assessed by Western blot analyses using cell lysates or membrane preparations of a panel of target sample/cell lines as described hereinabove.

Polynucleotides Identified by the Invention

The polynucleotides can be conjugated to a detectable marker, e.g., an enzymatic label or a radioisotope for detection of nucleic acid and/or expression of the gene in a cell. A wide variety of appropriate detectable markers are known in the art, including fluorescent, radioactive, enzymatic or other ligands, such as avidin/biotin, which are capable of giving a detectable signal. In preferred embodiments, one will likely desire to employ a fluorescent label or an enzyme tag, such as urease, alkaline phosphatase or peroxidase, instead of radioactive or other environmental undesirable reagents. In the case of enzyme tags, colorimetric indicator substrates are known which can be employed to provide a means visible to the human eye or spectrophotometrically, to identify specific hybridization with complementary nucleic acid-containing samples.

This invention further provides a method for detecting a single-stranded polynucleotide or its complement, by contacting target single-stranded polynucleotides with a labeled, single-stranded polynucleotide (a probe) under conditions permitting hybridization (preferably moderate or stringent hybridization conditions) of complementary single-stranded polynucleotides, or more preferably, under highly stringent hybridization conditions. Hybridized polynucleotide pairs are separated from un-hybridized, single-stranded polynucleotides. The hybridized polynucleotide pairs are detected using methods well known to those of skill in the art and set forth, for example, in Sambrook et al. (1989) supra.

The polynucleotides of this invention can be replicated using PCR. PCR technology is the subject matter of U.S. Pat. Nos. 4,683,195, 4,800,159, 4,754,065, and 4,683,202 and described in PCR: THE POLYMERASE CHAIN REACTION (Mullis et al. eds., Birkhauser Press, Boston (1994)) and references cited therein.

Alternatively, one of skill in the art can use the sequences provided herein and a commercial DNA synthesizer to replicate the DNA. Accordingly, this invention also provides a process for obtaining the polynucleotides of this invention by providing the linear sequence of the polynucleotide, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

RNA can be obtained by first inserting a DNA polynucleotide into a suitable host cell. The DNA can be inserted by any appropriate method, e.g., by the use of an appropriate gene delivery vehicle (e.g., liposome, plasmid or vector) or by electroporation. When the cell replicates and the DNA is transcribed into RNA; the RNA can then be isolated using methods well known to those of skill in the art, for example, as set forth in Sambrook et al. (1989) supra. For instance, MRNA can be isolated using various lytic enzymes or chemical solutions according to the procedures set forth in Sambrook et al. (1989) supra or extracted by nucleic-acid-binding resins following the accompanying instructions provided by manufactures.

It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. Preferably, a probe useful for detecting the aforementioned mRNA is at least about 80% identical to the homologous region of comparable size. More preferably, the probe is 85% identical to the corresponding gene sequence after alignment of the homologous region; even more preferably, it exhibits 90% identity.

These probes can be used in radioassays (e.g. Southern and Northern blot analysis) to detect or monitor various cells or tissue containing these cells. The probes also can be attached to a solid support or an array such as a chip for use in high throughput screening assays for the detection of expression of the gene corresponding to one or more polynucleotide(s) of this invention.

The invention further provides the isolated polynucleotide operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are well known in the art and commercially available. For general methodology and cloning strategies, see “Gene Expression Technology” (Goeddel ed., Academic Press, Inc. (1991)) and references cited therein and “Vectors: Essential Data Series” (Gacesa and Ramji, eds., John Wiley & Sons, N.Y. (1994)), which contains maps, functional properties, commercial suppliers and a reference to GenEMBL accession numbers for various suitable vectors. Preferably, these vectors are capable of transcribing RNA in vitro or in vivo.

Delivery Vehicles Comprising a Polynucleotide of the Invention

The present invention also provides delivery vehicles suitable for delivery of a polynucleotide of the invention into cells (whether in vivo, ex vivo, or in vitro). A polynucleotide of the invention can be contained within a cloning or expression vector. These vectors (especially expression vectors) can in turn be manipulated to assume any of a number of forms that may, for example, facilitate delivery to and/or entry into a cell.

Expression vectors containing these nucleic acids are useful to obtain host vector systems to produce proteins and polypeptides. It is implied that these expression vectors must be replicable in the host organisms either as episomes or as an integral part of the chromosomal DNA. Suitable expression vectors include plasmids, viral vectors, including adenoviruses, adeno-associated viruses, retroviruses, cosmids, etc. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells constructed using well known methods. See Sambrook, et al. (1989) supra. In addition to the use of viral vector for insertion of exogenous nucleic acid into cells, the nucleic acid can be inserted into the host cell by methods well known in the art such as transformation for bacterial cells; transfection using calcium phosphate precipitation for mammalian cells; or DEAE-dextran; electroporation; or microinjection. See Sambrook et al. (1989) supra for this methodology. Thus, this invention also provides a host cell, e.g., a mammalian cell, an animal cell (rat or mouse), a human cell, or a prokaryotic cell such as a bacterial cell, containing a polynucleotide encoding a protein or polypeptide or antibody.

When the vectors are used for gene therapy in vivo or ex vivo, a pharmaceutically acceptable vector is preferred, such as a replication-incompetent retroviral or adenoviral vector. Pharmaceutically acceptable vectors containing the nucleic acids of this invention can be further modified for transient or stable expression of the inserted polynucleotide.

Host Cells Comprising Polynucleotides of the Invention

The present invention further provides host cells comprising polynucleotides of the invention. Host cells containing the polynucleotides of this invention are useful for the recombinant replication of the polynucleotides and for the recombinant production of peptides of the invention. Alternatively, host cells comprising a polynucleotide of the invention may be used to induce an immune response in a subject in the methods described herein.

Host cells which are suitable for recombinant replication of the polynucleotides of the invention, and for the recombinant production of peptides of the invention can be prokaryotic or eukaryotic. Host systems are known in the art and need not be described in detail herein. Prokaryotic hosts include bacterial cells, for example E. coli, B. subtilis, and mycobacteria. Among eukaryotic hosts are yeast, insect, avian, plant, C. elegans (or nematode) and mammalian cells. These cells are cultured in conventional nutrient media modified as appropriate for inducing promoters, selecting transformants, or amplifying the genes encoding the desired sequences.

When the host cells are antigen presenting cells, they can be used to expand a population of immune effector cells such as tumor infiltrating lymphocytes which in turn are useful in adoptive immunotherapies. Antigen presenting cells are described in more detail below.

Host Cells Presenting Antigens of the Invention

The invention further provides isolated host cells comprising antigens identified by the methods of this invention. In some embodiments, these host cells present two or more peptides of the invention on the surface of the cell in the context of an MHC molecule such that the peptide can be recognized by an immune effector cell. Isolated host cells which present the polypeptides of this invention in the context of MHC molecules are further useful to expand and isolate a population of educated, antigen-specific immune effector cells. The immune effector cells, e.g., cytotoxic T lymphocytes, are produced by culturing naïve immune effector cells with antigen-presenting cells which present the polypeptides in the context of MHC molecules on the surface of the APCs. The population can be purified using methods known in the art, e.g., FACS analysis or FICOLL™ gradient. The methods to generate and culture the immune effector cells as well as the populations produced thereby also are the inventor's contribution and invention. Pharmaceutical compositions comprising the cells and pharmaceutically acceptable carriers are useful in adoptive immunotherapy. Prior to administration in vivo, the immune effector cells are screened in vitro for their ability to target cells.

In some of these embodiments, isolated host cells are APCs. APCs include, but are not limited to, dendritic cells (DCs), monocytes/macrophages, B lymphocytes or other cell type(s) expressing the necessary MHC/co-stimulatory molecules.

In some embodiments, the immune effector cells and/or the APCs are genetically modified. Using standard gene transfer, genes coding for co-stimulatory molecules and/or stimulatory cytokines can be inserted prior to, concurrent to or subsequent to expansion of the immune effector cells.

APCs can obtained from a variety of sources, including but not limited to, peripheral blood mononuclear cells (PBMC), whole blood or fractions thereof containing mixed populations, spleen cells, bone marrow cells, tumor infiltrating lymphocytes, cells obtained by leukapheresis, lymph nodes, e.g., lymph nodes draining from a tumor. Suitable donors include an immunized donor, a non-immunized (naïve) donor, treated or untreated donors. A “treated” donor is one that has been exposed to one or more biological modifiers. An “untreated” donor has not been exposed to one or more biological modifiers. APCs can also be treated in vitro with one or more biological modifiers.

The APCs are generally alive but can also be irradiated, mitomycin C treated, attenuated, or chemically fixed. Further, the APCs need not be whole cells. Instead, vesicle preparations of APCs can be used.

APCs can be genetically modified, i.e., transfected with a recombinant polynucleotide construct such that they express a polypeptide or an RNA molecule which they would not normally express or would normally express at lower levels. Examples of polynucleotides include, but are not limited to, those which encode an MHC molecule; a co-stimulatory molecule such as B7; and a peptide or polypeptide of the invention.

Cells which do not normally function in vivo in mammals as APCs can be modified in such a way that they function as APCs. A wide variety of cells can function as APCs when appropriately modified. Examples of such cells are insect cells, for example Drosophila or Spodoptera; and foster cells, such as the human cell line T2. For example, expression vectors which direct the synthesis of one or more antigen-presenting polypeptides, such as MHC molecules, optionally also accessory molecules such as B7, can be introduced into these cells to effect the expression on the surface of these cells antigen presentation molecules and, optionally, accessory molecules or functional portions thereof. Alternatively, antigen-presenting polypeptides and accessory molecules which can insert themselves into the cell membrane can be used. For example, glycosyl-phosphotidylinositol (GPI)-modified polypeptides can insert themselves into the membranes of cells. Hirose et al. (1995) Methods Enzymol. 250:582-614; and Huang et al. (1994) Immunity 1:607-613. Accessory molecules include, but are not limited to, co-stimulatory antibodies such as antibodies specific for CD28, CD80, or CD86; costimulatory molecules, including, but not limited to, B7.1 and B7.2; adhesion molecules such as ICAM-1 and LFA-3; and survival molecules such as Fas ligand and CD70. See, for example, PCT Publication No. WO 97/46256.

Foster antigen presenting cells are particularly useful as APCs. Foster APCs are derived from the human cell line 174xCEM.T2, referred to as T2, which contains a mutation in its antigen processing pathway that restricts the association of endogenous peptides with cell surface MHC class I molecules. Zweerink et al. (1993) J. Immunol. 150:1763-1771. This is due to a large homozygous deletion in the MHC class II region encompassing the genes TAP 1, TAP2, LMP 1, and LMP2, which are required for antigen presentation to MHC class 1-restricted CD8⁺ CTLs. In effect, only “empty” MHC class I molecules are presented on the surface of these cells. Exogenous peptide added to the culture medium binds to these MHC molecules provided that the peptide contains the allele-specific binding motif. These T2 cells are referred to herein as “foster” APCs. They can be used in conjunction with this invention to present antigen(s).

Transduction of T2 cells with specific recombinant MHC alleles allows for redirection of the MHC restriction profile. Libraries tailored to the recombinant allele will be preferentially presented by them because the anchor residues will prevent efficient binding to the endogenous allele.

High level expression of MHC molecules makes the APC more visible to the CTLs. Expressing the MHC allele of interest in T2 cells using a powerful transcriptional promoter (e.g., the CMV promoter) results in a more reactive APC (most likely due to a higher concentration of reactive MHC-peptide complexes on the cell surface).

The following is a brief description of two fundamental approaches for the isolation of APC. These approaches involve (1) isolating bone marrow precursor cells (CD34⁺) from blood and stimulating them to differentiate into APC; or (2) collecting the precommitted APCs from peripheral blood. In the first approach, the patient must be treated with cytokines such as GM-CSF to boost the number of circulating CD34⁺ stem cells in the peripheral blood.

The second approach for isolating APCs is to collect the relatively large numbers of precommitted APCs already circulating in the blood. Previous techniques for isolating committed APCs from human peripheral blood have involved combinations of physical procedures such as metrizamide gradients and adherence/nonadherence steps (Freudenthal et al. (1990) Proc. Natl. Acad. Sci. USA 87:7698-7702); Percoll gradient separations (Mehta-Damani et al. (1994) J. Immunol. 153:996-1003); and fluorescence activated cell sorting techniques (Thomas et al. (1993) J. Immunol. 151:6840-52).

One technique for separating large numbers of cells from one another is known as countercurrent centrifugal elutriation (CCE). In this technique, cells are subject to simultaneous centrifugation and a washout stream of buffer which is constantly increasing in flow rate. The constantly increasing countercurrent flow of buffer leads to fractional cell separations that are largely based on cell size.

In one aspect of the invention, the APC are precommitted or mature dendritic cells which can be isolated from the white blood cell fraction of a mammal, such as a murine, simian or a human (See, e.g., WO 96/23060). The white blood cell fraction can be from the peripheral blood of the mammal. This method includes the following steps: (a) providing a white blood cell fraction obtained from a mammalian source by methods known in the art such as leukapheresis; (b) separating the white blood cell fraction of step (a) into four or more subfractions by countercurrent centrifugal elutriation, (c) stimulating conversion of monocytes in one or more fractions from step (b) to dendritic cells by contacting the cells with calcium ionophore, GM-CSF and IL-13 or GM-CSF and IL-4, (d) identifying the dendritic cell-enriched fraction from step (c), and (e) collecting the enriched fraction of step (d), preferably at about 4° C. One way to identify the dendritic cell-enriched fraction is by fluorescence-activated cell sorting. The white blood cell fraction can be treated with calcium ionophore in the presence of other cytokines, such as recombinant (rh) rhIL-12, rhGM-CSF, or rhIL-4. The cells of the white blood cell fraction can be washed in buffer and suspended in Ca⁺⁺/Mg⁺⁺ free media prior to the separating step. The white blood cell fraction can be obtained by leukapheresis. The dendritic cells can be identified by the presence of at least one of the following markers: HLA-DR, HLA-DQ, or B7. 2, and the simultaneous absence of the following markers: CD3, CD 14, CD16, 56, 57, and CD 19, 20. Monoclonal antibodies specific to these cell surface markers are commercially available.

More specifically, the method requires collecting an enriched collection of white cells and platelets from leukapheresis that is then further fractionated by countercurrent centrifugal elutriation (CCE). Abrahamsen et al. (1991) J. Clin. Apheresis 6:48-53. Cell samples are placed in a special elutriation rotor. The rotor is then spun at a constant speed of, for example, 3000 rpm. Once the rotor has reached the desired speed, pressurized air is used to control the flow rate of cells. Cells in the elutriator are subjected to simultaneous centrifugation and a washout stream of buffer which is constantly increasing in flow rate. This results in fractional cell separations based largely but not exclusively on differences in cell size.

Quality control of APC and more specifically DC collection and confirmation of their successful activation in culture is dependent upon a simultaneous multi-color FACS analysis technique which monitors both monocytes and the dendritic cell subpopulation as well as possible contaminant T lymphocytes. It is based upon the fact that DCs do not express the following markers: CD3 (T cell); CD14 (monocyte); CD16, 56, 57 (NK/LAK cells); CD19, 20 (B cells). At the same time, DCs do express large quantities of HLA-DR, significant HLA-DQ and B7.2 (but little or no B7.1) at the time they are circulating in the blood (in addition they express Leu M7 and M9, myeloid markers which are also expressed by monocytes and neutrophils).

Once collected, the DC rich/monocyte APC fractions (usually 150 through 190) can be pooled and cryopreserved for future use, or immediately placed in short term culture.

Alternatively, others have reported that a method for upregulating (activating) dendritic cells and converting monocytes to an activated dendritic cell phenotype. This method involves the addition of calcium ionophore to the culture media convert monocytes into activated dendritic cells. Adding the calcium ionophore A23187, for example, at the beginning of a 24-48 hour culture period resulted in uniform activation and dendritic cell phenotypic conversion of the pooled “monocyte plus DC” fractions: characteristically, the activated population becomes uniformly CD14 (Leu M3) negative, and upregulates HLA-DR, HLA-DQ, ICAM-1, B7.1, and B7.2.

Specific combination(s) of cytokines have been used successfully to amplify (or partially substitute) for the activation/conversion achieved with calcium ionophore: these cytokines include but are not limited to purified or recombinant human (“rh”) rhGM-CSF, rhIL-2, and rhIL-4. Each cytokine when given alone is inadequate for optimal upregulation.

Presentation of Polypeptides by Antigen-Presenting Matrices

For use in immunomodulatory methods and diagnostic methods of the invention, an antigen-presenting matrix presents convergent antigenic peptide ligands of the invention bound to an MHC molecule. Any known method can be used to achieve presentation by an antigen-presenting matrix. The following are non-limiting examples of methods that can be used.

Polypeptides can be delivered to antigen-presenting cells as polypeptide or peptide or in the form of cDNA encoding the protein/peptide.

Another method to deliver a synthetic antigenic peptide epitope of the invention to an APC is by pulsing. Pulsing can be accomplished in vitro/ex vivo by exposing APCs to the antigenic polypeptide(s) or peptide(s) of this invention. The polypeptide(s) or peptide(s) are added to APCs at a concentration of 1-10 μm for approximately 3 hours. Pulsed APCs can subsequently be administered to the host via an intravenous, subcutaneous, intranasal, intramuscular or intraperitoneal route of delivery.

Polypeptides can also be delivered in vivo, for example, as part of a polypeptide or complexed with another macromolecule, with or without adjuvant via the intravenous, subcutaneous, intranasal, intramuscular or intraperitoneal route of delivery.

Various other techniques can be used, including the following. Paglia et al. (1996) J. Exp. Med. 183:317-322 has shown that APC incubated with whole protein in vitro are recognized by MHC class I-restricted CTLs, and that immunization of animals with these APCs led to the development of antigen-specific CTLs in vivo. In addition, several different techniques have been described which lead to the expression of antigen in the cytosol of APCs, such as DCs. These include (1) the introduction into the APCs of RNA isolated from tumor cells, (2) infection of APCs with recombinant vectors to induce endogenous expression of antigen, and (3) introduction of tumor antigen into the DC cytosol using liposomes. (See Boczkowski et al. (1996) J. Exp. Med. 184:465-472; Rouse et al. (1994) J. Virol. 68:5685-5689; and Nair et al. (1992) J. Exp. Med. 175:609-612).

Another method which can be used is termed “painting.” It has been demonstrated that glycosyl-phosphotidylinositol (GPI)-modified proteins possess the ability to reincorporate themselves back into cell membranes after purification. Hirose et al. (1995) Methods Enzymol. 250:582-614; Medof et al., (1984) J. Exp. Med. 160:1558-1578; Medof(1996) FASEB J. 10:574-586; and Huang et al. (1994) Immunity 1:607-613 have exploited this property in order to create APCs of specific composition for the presentation of antigen to CTLs. They devised expression vectors for β2-microglobulin and the HLA-A2.1 allele. The proteins were expressed in Schneider S2 Drosophila melanogaster cells, known to support GPI-modification. After purification, the proteins could be incubated together with a purified antigenic peptides which resulted in a trimolecular complex capable of efficiently inserting itself into the membranes of autologous cells. In essence, these protein mixtures were used to “paint” the APC surface, conferring the ability to stimulate a CTL clone that was specific for the antigenic peptide. Cell coating was shown to occur rapidly and to be protein concentration dependent. This method of generating APCs bypasses the need for gene transfer into the APC and permits control of antigenic peptide densities at the cell surfaces.

Immune Effector Cells

The present invention makes use of the above-described compositions including APCs, to stimulate production of an enriched population of antigen-specific immune effector cells. Accordingly, the present invention provides a population of cells enriched in educated, antigen-specific immune effector cells, specific for an antigenic peptide of the invention. These cells can cross-react with (bind specifically to) antigenic determinants (epitopes) on natural (endogenous) antigens. In some embodiments, the natural antigen is on the surface of tumor cells and the educated, antigen-specific immune effector cells of the invention suppress growth of the tumor cells. When APCs are used, the antigen-specific immune effector cells are expanded at the expense of the APCs, which die in the culture. The process by which naïve immune effector cells become educated by other cells is described essentially in Coulie (1997) Molec. Med. Today 3:261-268.

The APCs prepared as described above are mixed with naïve immune effector cells. Preferably, the cells may be cultured in the presence of a cytokine, for example IL-2. Because dendritic cells secrete potent immunostimulatory cytokines, such as IL-12, it may not be necessary to add supplemental cytokines during the first and successive rounds of expansion. In any event, the culture conditions are such that the antigen-specific immune effector cells expand (i.e. proliferate) at a much higher rate than the APCs. Multiple infusions of APCs and optional cytokines can be performed to further expand the population of antigen-specific cells.

In one embodiment, the immune effector cells are T cells. In a separate embodiment, the immune effector cells can be genetically modified by transduction with a transgene coding for example, IL-2, IL-11 or IL-13. Methods for introducing transgenes in vitro, ex vivo and in vivo are well known in the art. See Sambrook, et al. (1989) supra.

An effector cell population suitable for use in the methods of the present invention can be autogeneic or allogeneic, preferably autogeneic. When effector cells are allogeneic, preferably the cells are depleted of alloreactive cells before use. This can be accomplished by any known means, including, for example, by mixing the allogeneic effector cells and a recipient cell population and incubating them for a suitable time, then depleting CD69⁺ cells, or inactivating alloreactive cells, or inducing anergy in the alloreactive cell population.

Hybrid immune effector cells can also be used. Immune effector cell hybrids are known in the art and have been described in various publications. See, for example, International Patent Application Nos. WO 98/46785 and WO 95/16775.

The effector cell population can comprise unseparated cells, i.e., a mixed population, for example, a PBMC population, whole blood, and the like. The effector cell population can be manipulated by positive selection based on expression of cell surface markers, negative selection based on expression of cell surface markers, stimulation with one or more antigens in vitro or in vivo, treatment with one or more biological modifiers in vitro or in vivo, subtractive stimulation with one or more antigens or biological modifiers, or a combination of any or all of these.

Effector cells can obtained from a variety of sources, including but not limited to, PBMC, whole blood or fractions thereof containing mixed populations, spleen cells, bone marrow cells, tumor infiltrating lymphocytes, cells obtained by leukapheresis, biopsy tissue, lymph nodes, e.g., lymph nodes draining from a tumor. Suitable donors include an immunized donor, a non-immunized (naïve) donor, a treated or untreated donors. A “treated” donor is one that has been exposed to one or more biological modifiers. An “untreated” donor has not been exposed to one or more biological modifiers.

Methods of extracting and culturing effector cells are well known. For example, effector cells can be obtained by leukapheresis, mechanical apheresis using a continuous flow cell separator. For example, lymphocytes and monocytes can be isolated from the buffy coat by any known method, including, but not limited to, separation over Ficoll-Hypaque™ gradient, separation over a Percoll gradient, or elutriation. The concentration of Ficoll-Hypaque™ can be adjusted to obtain the desired population, for example, a population enriched in T cells. Other methods based on affinity are known and can be used. These include, for example, fluorescence-activated cell sorting (FACS), cell adhesion, magnetic bead separation, and the like. Affinity-based methods may utilize antibodies, or portions thereof, which are specific for cell-surface markers and which are available from a variety of commercial sources, including, the American Type Culture Collection (Manassas, Md.). Affinity-based methods can alternatively utilize ligands or ligand analogs, of cell surface receptors.

The effector cell population can be subjected to one or more separation protocols based on the expression of cell surface markers. For example, the cells can be subjected to positive selection on the basis of expression of one or more cell surface polypeptides, including, but not limited to, “cluster of differentiation” cell surface markers such as CD2, CD3, CD4, CD8, TCR, CD45, CD45RO, CD45RA, CD11b, CD26, CD27, CD28, CD29, CD30, CD31, CD40L; other markers associated with lymphocyte activation, such as the lymphocyte activation gene 3 product (LAG3), signaling lymphocyte activation molecule (SLAM), T1/ST2; chemokine receptors such as CCR3, CCR4, CXCR3, CCR5; homing receptors such as CD62L, CD44, CLA, CD146, a4b7, aEb7; activation markers such as CD25, CD69 and OX40; and lipoglycans presented by CD1. The effector cell population can be subjected to negative selection for depletion of non-T cells and/or particular T cell subsets. Negative selection can be performed on the basis of cell surface expression of a variety of molecules, including, but not limited to, B cell markers such as CD19, and CD20; monocyte marker CD14; the NK cell marker CD56.

An effector cell population can be manipulated by exposure, in vivo or in vitro, to one or more biological modifiers. Suitable biological modifiers include, but are not limited to, cytokines such as IL-2, IL-4, IL-10, TNF-α, IL-12, IFN-γ; non-specific modifiers such as phytohemagglutinin (PHA), phorbol esters such as phorbol myristate acetate (PMA), concanavalin-A, and ionomycin; antibodies specific for cell surface markers, such as anti-CD2, anti-CD3, anti-IL2 receptor, anti-CD28; chemokines, including, for example, lymphotactin. The biological modifiers can be native factors obtained from natural sources, factors produced by recombinant DNA technology, chemically synthesized polypeptides or other molecules, or any derivative having the functional activity of the native factor. If more than one biological modifier is used, the exposure can be simultaneous or sequential.

The present invention provides compositions comprising immune effector cells, which may be T cells, enriched in antigen-specific cells. By “enriched” is meant that a cell population is at least about 50-fold, more preferably at least about 500-fold, and even more preferably at least about 5000-fold or more enriched from an original naive cell population. The proportion of the enriched cell population which comprises antigen-specific cells can vary substantially, from less than 10% up to 100% antigen-specific cells. If the cell population comprises at least 50%, preferably at least 70%, more preferably at least 80%, and even more preferably at least 90%, antigen-specific immune effector cells, specific for a peptide of the invention, then the population is said to be “substantially pure.” The percentage which are antigen-specific can readily be determined, for example, by a ³H-thymidine uptake assay in which the effector cell population (for example, a T-cell population) is challenged by an antigen-presenting matrix presenting an antigenic peptide of the invention.

Isolated Antibodies and Derivative Antibodies

Serum antibodies, monoclonal antibodies and antibody derivatives are within the scope of this invention. The antibody compositions recognize the polypeptides identified by practicing the methods of the invention. Such antibodies include polyclonal and monoclonal antibodies. Laboratory methods for producing polyclonal antibodies and monoclonal antibodies, as well as deducing their corresponding nucleic acid sequences, are known in the art, see Harlow and Lane (1988) and (1999) supra and Sambrook, et al. (1989) supra. The monoclonal antibodies of this invention can be biologically produced by introducing protein or a fragment thereof into an animal, e.g., a mouse or a rabbit. The antibody producing cells in the animal are isolated and fused with myeloma cells or heteromyeloma cells to produce hybrid cells or hybridomas. Accordingly, the hybridoma cells producing the monoclonal antibodies of this invention also are provided.

Thus, using the protein or fragment thereof, and well known methods, one of skill in the art can produce and screen the hybridoma cells and antibodies of this invention for antibodies having the ability to bind the proteins or polypeptides.

If a monoclonal antibody being tested binds with the protein or polypeptide, then the antibody being tested and the antibodies provided by the hybridomas of this invention are equivalent. It also is possible to determine, without undue experimentation, whether an antibody has the same specificity as the monoclonal antibody of this invention by determining whether the antibody being tested prevents a monoclonal antibody of this invention from binding the protein or polypeptide with which the monoclonal antibody is normally reactive. If the antibody being tested competes with the monoclonal antibody of the invention as shown by a decrease in binding by the monoclonal antibody of this invention, then it is likely that the two antibodies bind to the same or a closely related epitope. Alternatively, one can pre-incubate the monoclonal antibody of this invention with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this invention.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. USA 82:8653 or Spira et al. (1984) J. Immunol. Meth. 74:307.

This invention also provides biological active fragments of the polyclonal and monoclonal antibodies described above. These “antibody fragments” retain some ability to selectively bind with its antigen or immunogen. Such antibody fragments can include, but are not limited to: 1) Fab, 2) Fab′, 3) F(ab′)₂, 4) Fv, and 5) single chain antibodies (“SCA”).

A specific example of “a biologically active antibody fragment” is a complementarity determining region (CDR) of the antibody. Methods of making these fragments are known in the art, see for example, Harlow and Lane (1988) and (1999) supra.

The antibodies of this invention also can be modified to create chimeric and humanized antibodies (Oi et al. (1986) BioTechniques 4(3):214). Chimeric antibodies are those in which the various domains of the antibodies' heavy and light chains are coded for by DNA from more than one species.

The isolation of other hybridomas secreting monoclonal antibodies with the specificity of the monoclonal antibodies of the invention can also be accomplished by one of ordinary skill in the art by producing anti-idiotypic antibodies (Herlyn et al. (1986) Science 232:100). An anti-idiotypic antibody is an antibody which recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

As used in this invention, the term “epitope” is meant to include any determinant having specific affinity for the monoclonal antibodies of the invention. Epitopic determinants usually consist of chemically active surface groupings of molecules such as amino acids or sugar side chains and usually have specific three dimensional structural characteristics, as well as specific charge characteristics.

The antibodies and/or antigens (polypeptides) of this invention can be linked to a detectable agent or label. There are many different labels and methods of labeling known to those of ordinary skill in the art.

The coupling of antibodies to low molecular weight haptens can increase the sensitivity of the assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See Harlow and Lane (1988) and (1999) supra.

The antibodies of the invention also can be bound to many different carriers. Thus, this invention also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

Compositions containing the antibodies, fragments thereof or cell lines which produce the antibodies, are encompassed by this invention. When these compositions are to be used pharmaceutically, they are combined with a pharmaceutically acceptable carrier.

Compositions containing antibodies or antigens of the invention are useful to detect and isolate specific antigenic polypeptides, antibody-reactive peptide epitopes and therapeutic antibody molecules. These compositions have a variety of uses for diagnosing and inhibiting pathological cells. For example, antigen-reactive antibodies can be generated by immunizing an animal with the antigenic polypeptide using methods well known in the art. It is also desirable to prepare a monoclonal antibody for administration to a subject. For use with human subjects, methods have now been established to produce “humanized antibodies” where species specific portions of the antibody molecule have been converted to sequences characteristic of human antibodies. Such molecules function more effectively when administered to a human subject.

Diagnostic antibodies are useful for detecting a pathological cell and a variety of alternative techniques for labeling and detecting these antibodies have been established. For example, the antibody can be conjugated to a radioactive isotope that can be localized in the subject following administration of the antibody. It is a specific purpose of the present invention to provide an improved means for obtaining novel antigenic peptides and antibodies that recognize these peptides for use as diagnostic agents.

Therapeutic antibodies can also be administered to a subject to inhibit the progression of disease. In a human subject it is desirable to administer a humanized monoclonal antibody for this purpose. The antibody can confer a passive immunity wherein it inhibits disease by binding to antigens in the target pathological tissue and inducing complement mediated cytotoxicity, antibody-directed cytotoxicity, or interference with receptor-ligand interactions. Alternatively, administration of an antibody to a subject can vaccinate against disease by inducing an anti-idiotype immune response. For use in a human subject a monoclonal antibody such as a mouse monoclonal antibody is effective for this purpose.

The antigenic polypeptides identified by practicing the methods of the invention are also useful as therapeutic agents when administered to a subject or useful to educate naïve immune effector cells. Such polypeptides can be formulated with an adjuvant and administered as a vaccine to induce an immune response against the pathological target tissue. Such antigenic polypeptides can also be administered ex vivo, for example to dendritic cells isolated from the subject. The antigen pulsed dendritic cells can then be expanded in culture and returned to the subject to perform adoptive immunotherapy.

Similar methods can be practiced using synthetic and natural peptides comprising the antigenic epitopes identified by the methods of the invention. Such peptides can be administered in vivo or ex vivo. They may also be delivered using a gene delivery vehicle that comprises a polynucleotide sequence encoding the appropriate amino acid sequence of the antigen and epitope. A variety of formats and formulations for vaccinating subjects with recombinant vaccines are well known to those skilled in the art.

Compositions of the Invention

This invention also provides compositions containing any of the above-mentioned peptides, polypeptides, polynucleotides, antigen-presenting matrices, vectors, cells, antibodies and fragments thereof, and an acceptable solid or liquid carrier. When the compositions are used pharmaceutically, they are combined with a “pharmaceutically acceptable carrier” for diagnostic and therapeutic use. These compositions also can be used for the preparation of medicaments for the diagnostic and immunomodulatory methods of the invention.

Diagnostic Methods

The present invention provides diagnostic methods using polypeptides and antibodies of the invention. The methods can be used to detect the presence of an antigen-specific CD4⁺ or CD8⁺ T cell which binds the polypeptide of the invention.

The diagnostic methods of the invention include: (1) assays to predict the efficacy of a polypeptide of the invention; (2) assays to determine the precursor frequency (i.e., the presence and number of) of immune effector cells specific for a polypeptide and/or its natural counterpart; and (3) assays to determine the efficacy of a polypeptide or antibody once in an immunomodulatory method of the invention. Antibodies also can be used to identify the cell surface ligand against which the antibody has been raised.

Diagnostic methods of the invention are generally carried out under suitable conditions and for a sufficient time to allow specific binding to occur between a polypeptide and an immune effector molecule, such as a TCR, on the surface of an immune effector cell, such as a CD4+ or CD8+ T cell or antibody. “Suitable conditions” and “sufficient time” are generally conditions and times suitable for specific binding. Suitable conditions occur between about 4° C. and about 40° C., preferably between about 4° C. and about 37° C., in a buffered solution, and within a pH range of between 5 and 9. A variety of buffered solutions are known in the art, can be used in the diagnostic methods of this invention, and include, but are not limited to, phosphate-buffered saline. Sufficient time for binding and response will generally be between about 1 second and about 24 hours after exposure of the sample to the convergent antigenic peptide ligand.

In some embodiments, the invention provides diagnostic assays to predict the efficacy of a polypeptide of the invention. In some of these embodiments, defined T cell epitopes are used to clinically characterize tumors and viral pathogens in order to determine in advance the predicted efficacy of an in vivo vaccine trial. This can be achieved by a simple proliferation assay of a patient's peripheral blood mononuclear cells using defined T cell epitopes as stimulators. Polypeptides that elicit a response are viable vaccine candidates for that patient.

In other embodiments, assays are provided to determine the precursor frequency (i.e., the presence and number of) of resting (naïve) immune effector cells specific for a polypeptide of this invention and which therefore have the potential to become activated. In these embodiments, an antigen-presenting cell bearing on its surface a polypeptide of the invention is used to detect the presence of immune effector cells in a biological sample. A functional assay is used to determine (and quantitate) the antigen-specific immune effector cells. As an illustrative example, PBMCs are isolated from a subject with a tumor. A sample of these PBMCs is cultured together for a suitable time with the target cells from the same subject. Functional assays include, but are not limited to, immune effector cell proliferation, cytokine production, specific lysis of an APC.

In other embodiments, the efficacy of an immunomodulatory method, including immunomodulatory methods of the invention, in modulating an immune response to a polypeptide of the invention can be tested using diagnostic assays of the invention. These diagnostic assays are also useful to confirm biological activity of the polypeptide or monitor the efficacy of an immunotherapeutic agent. In some of these embodiments, the method allows detection of immune effector cells, which may be activated CD4⁺ or CD8⁺ T cells, which have become activated or anergized as a result of exposure to a polypeptide of the invention. A sample containing cells from a subject can be tested for the presence of CD4⁺ or CD8⁺ T cells which have become activated or anergized as a result of binding to a given polypeptide of the invention.

Immunomodulatory Methods

The invention provides methods of modulating an immune response in an individual to a polypeptide of the invention. Immunomodulatory methods of the invention include methods that result in induction or increase, as well as methods that result in suppression or reduction, of an immune response in a subject, and comprise delivering to the subject an effective amount of a peptide (or any immunomodulatory agent) of the invention in formulations and/or under conditions that result in the desired effect on an immune response (or lack thereof) to the peptide. Immunomodulatory methods of the invention include vaccine methods, adoptive immunotherapy, and methods to induce T cell anergy.

An “immunomodulatory agent” for use in the methods of the invention is a molecule, a macromolecular complex, or a cell that modulates an immune response and encompasses: a polypeptide of the invention alone or in any of a variety of formulations described herein; a polypeptide comprising a polypeptide; a polynucleotide encoding a peptide or polypeptide of the invention; a polypeptide bound to MHC Class I which in turn is bound to an antigen-presenting matrix, including an APC (in the presence or absence of co-stimulatory molecule(s)); a polypeptide or antibody or a derivative thereof covalently or non-covalently complexed to another molecule(s) or macromolecular structure; and an educated, antigen-specific immune effector cell which is specific for a polypeptide of the invention.

Various methods are known to evaluate T cell activation. CTL activation can be detected by any known method, including but not limited to, tritiated thymidine incorporation (indicative of DNA synthesis), and examination of the population for growth or proliferation, e.g., by identification of colonies. Alternatively, the tetrazolium salt MTT (3-(4,5-dimethyl-thazol-2-yl)-2,5-diphenyl tetrazolium bromide) may be added. Mossman (1983) J. Immunol. Methods 65:55-63; Niks and Otto (1990) J. Immunol. Methods 130:140-151. Succinate dehydrogenase, found in mitochondria of viable cells, converts the MTT to formazan blue. Thus, concentrated blue color would indicate metabolically active cells. In yet another embodiment, incorporation of radiolabel, e.g., tritiated thymidine, may be assayed to indicate proliferation of cells. Similarly, protein synthesis may be shown by incorporation of ³⁵S-methionine. In still another embodiment, cytotoxicity and cell killing assays, such as the classical chromium release assay, may be employed to evaluate epitope-specific CTL activation. To detect activation of CD4⁺ T cells, any of a variety of methods can be used, including, but not limited to, measuring cytokine production; and proliferation, for example, by tritiated thymidine incorporation

Release of ⁵¹Cr from labeled target cells is a standard assay which can be used to assess the number of peptide-specific CTLs in a biological sample. Tumor cells, or APCs of the invention, are radiolabeled as targets with about 200 μCi of Na₂ ⁵¹CrO₄ for 60 minutes at 37° C., followed by washing. T cells and target cells (˜1×10⁴/well) are then combined at various effector-to-target ratios in 96-well, U-bottom plates. The plates are centrifuged at 100×g for 5 minutes to initiate cell contact, and are incubated for 4-16 hours at 37° C. with 5% CO₂. Release of ⁵¹Cr is determined in the supernatant, and compared with targets incubated in the absence of T cells (negative control) or with 0.1% TRITON™ X-100 (positive control). See, e.g., Mishell and Shiigi, eds. Selected Methods in Cellular Immunology (1980) W. H. Freeman and Co.

The formulation of a polypeptide of the invention will vary, depending on the desired result. In general, peptides presented on an antigen-presenting matrix by a Class I MHC molecule, together with the appropriate co-stimulatory molecules, will result in induction of an immune response to the polypeptide. An anergic (or unresponsive) state may be induced in T lymphocytes by presentation of an antigen by an antigen-presenting matrix (which may be an APC) which contains appropriate MHC molecules on its surface, but which lacks the appropriate co-stimulatory molecules. Any of the various formulations described herein can be used.

Polynucleotides of the invention can be administered in a gene delivery vehicle or by inserting into a host cell which in turn recombinantly transcribes, translates and processed the encoded polypeptide. Isolated host cells containing a polynucleotide of the invention in a pharmaceutically acceptable carrier can be combined with appropriate and effective amount of an adjuvant, cytokine or co-stimulatory molecule for an effective vaccine regimen. In some embodiments, the host cell is an APC, such as a dendritic cell. The host cell can be further modified by inserting of a polynucleotide coding for an effective amount of either or both of a cytokine a co-stimulatory molecule.

The methods of this invention can be further modified by co-administering an effective amount of a cytokine or co-stimulatory molecule to the subject.

The agents provided herein as effective for their intended purpose can be administered to subjects having a disease to be treated with an immunomodulatory method of the invention or to individuals susceptible to or at risk of developing such a disease. When the agent is administered to a subject such as a mouse, a rat or a human patient, the agent can be added to a pharmaceutically acceptable carrier and systemically or topically administered to the subject. Therapeutic amounts can be empirically determined and will vary with the pathology or condition being treated, the subject being treated and the efficacy and toxicity of the therapy.

The amount of a polypeptide, antibody, antibody derivative, host cell or immune effector cell of the invention will vary depending, in part, on its intended effect, and is ultimately at the discretion of the medical or veterinary practitioner. The factors to be considered include the condition being treated, the route of administration, and nature of the formulation, the mammal's body weight, surface area, age, and general condition and the particular peptide to be administered. A suitable effective dose of an immune effector cell of the invention generally lies in the range of from about 10² to about 10⁹ cells per administration. Cells can be administered once, followed by monitoring of the clinical response, such as diminution of disease symptoms or tumor mass. Administration may be repeated on a monthly basis, for example, or as appropriate. Those skilled in the art will appreciate that an appropriate administrative regimen would be at the discretion of the physician or veterinary practitioner.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The agents and compositions of the present invention can be used in the manufacture of medicaments and for the treatment of humans and other animals by administration in accordance with conventional procedures, such as an active ingredient in pharmaceutical compositions.

More particularly, an agent of the present invention also referred to herein as the active ingredient, may be administered for therapy by any suitable route including nasal, topical (including transdermal, aerosol, buccal and sublingual), parenteral (including subcutaneous, intramuscular, intravenous and intradermal) and pulmonary. It will also be appreciated that the preferred route will vary with the condition and age of the recipient, and the disease or condition being treated.

Vaccines for Cancer Treatment and Prevention

In one embodiment, immunomodulatory methods of the present invention comprise vaccines for cancer treatment. Cancer cells contain many new antigens potentially recognizable by the immune system. Given the speed with which epitopes can be identified, custom anticancer vaccines can be generated for affected individuals by isolating TILs from patients with solid tumors, determining their MHC restriction, and assaying these CTLs against the appropriate library for reactive epitopes. These vaccines will be both treatments for affected individuals as well as preventive therapy against recurrence (or establishment of the disease in patients which present with a familial genetic predisposition to it). Inoculation of individuals who have never had the cancer is expected to be quite successful as preventive therapy, even though a tumor antigen-specific CTL response has not yet been elicited, because in most cases high affinity peptides seem to be immunogenic suggesting that holes in the functional T cell repertoire, if they exist, may be relatively rare. Sette et al. (1994) J. Immunol., 153:5586-5592. In mice, vaccination with appropriate epitopes not only eliminates established tumors but also protects against tumor re-establishment after inoculation with otherwise lethal doses of tumor cells. Bystryn et al. (1993) supra.

Recent advances in vaccine adjuvants provide effective means of administering peptides so that they impact maximally on the immune system. Del-Giudice (1994) Experientia 50:1061-1066. These peptide vaccines will be of great value in treating metastatic tumors that are generally unresponsive to conventional therapies. Tumors arising from the homozygous deletion of recessive oncogenes are less susceptible to elimination by a humoral (antibody) response and would thus be treated more effectively by eliciting a cellular, CTL response.

Vaccines for Diseases Caused by Pathogenic Organisms

The polypeptides of the present invention are also useful in methods to induce (or increase, or enhance) an immune response to a pathogenic organism. These include pathogenic viruses, bacteria, and protozoans.

Viral infections are ideal candidates for immunotherapy. Immunological responses to viral pathogens are sometimes ineffective as in the case of the lentiviruses such as HIV which causes AIDS. The high rates of spontaneous mutation make these viruses elusive to the immune system. However, a saturating profile of CTL epitopes presented on infected cells will identify shared antigens among different serotypes in essential genes that are largely intolerant to mutation which would allow the design of more effective vaccines.

Adoptive Immunotherapy Methods

The expanded populations of antigen-specific immune effector cells and APCs of the present invention find use in adoptive immunotherapy regimes and as vaccines. These compositions are useful to confirm therapeutic and diagnostic efficacy.

Adoptive immunotherapy methods involve, in one aspect, administering to a subject a substantially pure population of educated, antigen-specific immune effector cells made by culturing naïve immune effector cells with APCs as described above. In some embodiments, the APCs are dendritic cells.

In one embodiment, the adoptive immunotherapy methods described herein are autologous. In this case, the APCs are made using parental cells isolated from a single subject. The expanded population also employs T cells isolated from that subject. Finally, the expanded population of antigen-specific cells is administered to the same patient.

In a further embodiment, APCs or immune effector cells are administered with an effective amount of a stimulatory cytokine, such as IL-2 or a co-stimulatory molecule.

Methods of Inducing T Cell Anergy

Antigenic polypeptides isolated by the methods of the present invention are useful in methods to induce T cell anergy. Disorders which can be treated using these methods include autoimmune disorders, allergies, and allograft rejection.

Autoimmune disorders are diseases in which the body's immune system responds against self tissues. They include most forms of arthritis, ulcerative colitis, and multiple sclerosis. Convergent antigenic peptide ligands corresponding to endogenous elements that are recognized as foreign can be used in the development of treatments using gene therapy or other approaches. For example, synthetic CTL epitopes, which can act as “suicide substrates” for CTLs that mediate autoimmunity, can be designed as described above. That is to say, peptides which have a high affinity for the MHC allele but fail to activate the TCR could effectively mask the cellular immune response against cells presenting the antigen in question. In support of this approach, it is believed that the long latency period of the HIV virus is due to an antiviral immune response and a mechanism by which the virus finally evades the immune system is by generating epitopes that occupy the MHC molecules but do not stimulate a TCR lytic response, inducing specific T cell anergy. Klenerman et al. (1995) Eur. J. Immunol. 25:1927-1931.

In vitro stimulation of T cells through the complex of T cell-antigen receptor and CD3 alone in the absence of other signals, induces T cell anergy or paralysis. T cell activation as measured by interleukin-2 production and proliferation in vitro requires both antigenic and co-stimulatory signals engendered by cell to cell interactions among antigen-specific T cells and antigen presenting cells. Various interactions of these CD2 proteins on the T-cell surface with CD58 (LFA-3) proteins and antigen-presenting cells, those of CD11a/CD18 (LFA-1) proteins with CD54 (ICAM-1) proteins and those of CD5 proteins with CD72 proteins can impart such a co-stimulatory signal in vitro. Cytokines derived from antigen-presenting cells (e.g., interleukin-1 and interleukin-6) can also provide co-stimulatory signals that result in T-cell activation in vitro. The delivery of both antigenic and co-stimulatory signals leads to stable transcription of the interleukin-2 gene and other pivotal T cell-activation genes. The foregoing co-stimulatory signals depend on protein kinase C and calcium. Potent antigen presenting cells express CD80 (B7 and BB1) and other related surface proteins and many T cells express B7 binding proteins, namely CD28 and CTLA-4 proteins. Binding of CD80 by CD28 and CDLA-4 stimulates a T cell co-stimulatory pathway that is independent of protein kinase C and calcium leading to vigorous T cell proliferation. The stimulation of B cells also depends on the interaction between the specific antigen and the cell-surface immunoglobulin. T cell derived cytokines (e.g., interleukins 1 and 4), physical contact between T cells and B cells through specific pairs of receptors and co-receptors, or both, provide the signal or signals essential for B cell stimulation.

Conventional routes of administration are used. A T-cell stimulating or anergy producing amount (or therapeutically effective amount as described above) of an immunotherapeutic antigen-superantigen polymer according to the invention is contacted with the target cells. By “T-cell anergy effective amount” is intended an amount which is effective in producing a statistically significant inhibition of a cellular activity mediated by a TCR. This may be assessed in vitro using T-cell activation tests. Typically, T-cell anergy or activation is assayed by tritiated thymidine incorporation in response to specific antigen.

One way in which T cell anergy can be induced is to present to a T cell an antigen-presenting matrix which presents a polypeptide of the invention in an MHC Class I molecule, but which lack co-stimulatory molecules necessary to activate the T cell. For example, a cell other than a normal antigen presenting cell (APC), which has been transfected with MHC antigen to which a selected T cell clone is restricted, can be used. Resting T cells are provided with an appropriate peptide recognized by the resting T cells in the context of the MHC transfected into a cellular host other than an APC. The MHC is expressed as a result of introduction into a mammalian cell other than an antigen presenting cell of genes constitutively expressing an MHC Class I molecule together with invariant chain. Importantly, these cells do not provide other proteins, either cell surface proteins or secreted proteins, associated with antigen presenting cells, which together with the MHC and peptide result in co-stimulatory signals.

To determine whether anergy has been induced, the T cells to be tested can be cultured together with an which presents the antigens of the invention in an MHC Class I molecule together with co-stimulatory molecules necessary to activate the T cell. The cultures are incubated for about 48 hours, then pulsed with tritiated thymidine and incorporation measured about 18 hours later. The absence of incorporation above control levels, where the T-cells are presented with antigen presenting cells which do not stimulate the T cells, either due to using an MHC to which the T cells are not restricted or using a peptide to which the T cells are not sensitive, is indicative of an absence of activation. One may use other conventional assays to determine the extent of activation, such as assaying for IL-2,-3, or -4, cell surface proteins associated with activation, e.g. CD71 or other convenient techniques. Another method is to determine the expression of a protein which is expressed on quiescent T cells, but not on anergic T cells. U.S. Pat. No. 5,747,299.

The following examples are intended to illustrate but not limit the invention.

Experimental Methods

Method for Isolating Apical and Basolateral Cell Membrane Proteins

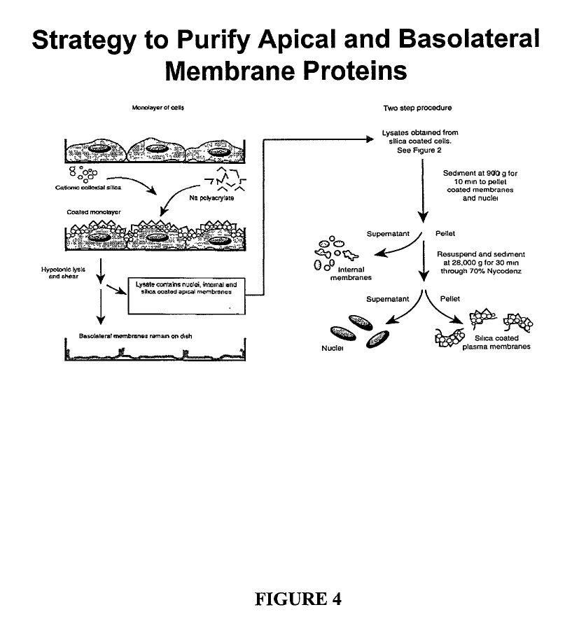

FIG. 4 shows a schematic for isolation of apical and basolateral cell membranes. This modification of the method disclosed in Chaney and Jacobsen (1983) J. Biol. Chem. 258:10062-10072, enriches for apical and basolateral membrane proteins as compared to the yields of a whole cell lysate preparation. First, a monolayer of target cells are coated with an “activated” cationic colloidal silica particle slurry that binds to the plasma membrane. By “activated” it is intended that the slurry acquire a positive charge (i.e., become cationic) through coating the silica particles with aluminum chlorohydroxide. Next, an anionic polymer, such as sodium polyacrylate, is added to crosslink the cationic silica particles, generating a structurally stable pellicle on the outside of the plasma membranes. The cells are then contacted with a hypotonic imidazole-based buffer to gently swell and rupture the cells. The resulting lysate contains nuclei, internal and silica coated apical membranes; while the basolateral membrane remains attached to the cell culture plate. The aqueous lysate is sedimented at 900 g for 10 min to pellet the coated membranes and nuclei. The resulting supernatant containing internal membranes is discarded. The pelleted nuclei and membrane are resuspended and further sedimented at 28,000 g for 30 min. through 70% Nycodenz (Sigma, St. Louis, Mo.) and the resulting supernatant containing nuclei is discarded. The remaining pellet contains silica coated apical membranes. The isolated apical membrane preparation may be used alone or in combination with the basolateral membrane preparation (which can be readily removed from the culture plate using standard cell culture techniques).

Antibody Elution from an Immunoblot

In order to conduct further analyses with a serum antibody that is bound to an antigen of interest on a solid support, the antibody must be removed in a manner so as to retain its functionality. In order to isolate the functional antibody, the portion of the solid support (e.g. nitrocellulose) to which the antibody is bound is excised and the antibody is eluted off of the solid support using a method adapted from Maa, J. S., Rodriguez, et al. (1990) J. Biol. Chem. 265:1569-1577.

After a band of interest is identified by the described Western blotting technique, a prep-scale gel containing as much of the target antigen as possible is prepared and transferred to nitrocellulose. The blot is incubated with the appropriate serum or antibody and washed according to the standard Western blotting protocol. Antibody reactivities (i.e., bands) present on the blot are visualized on film by standard techniques (i.e., radioactive labeling, ECL, etc.)

After aligning the film with the nitrocellulose membrane, the fragment of nitrocellulose containing the band of interest is excised with a scalpel, placed into a tube containing 100 mM glycine pH 2.5 buffer and incubated at room temperature for 5 min. The fragment of nitrocellulose is then removed from the tube and the buffer (containing the eluted antibody) is neutralized with 1/10 volume of 1M Tris pH 8.0. After neutralization, BSA is added to the buffer (eluate) to a final concentration of 1%.

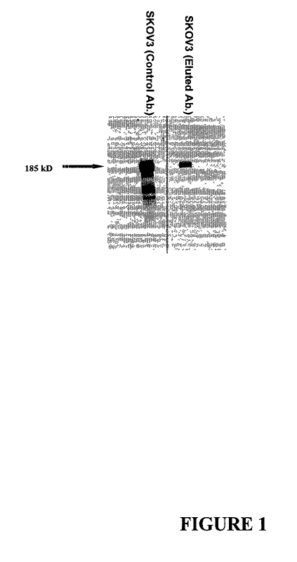

FIG. 1 shows that antibody eluted by the above protocol retains its specific antigen binding property. The blot on the right was generated with anti-Her2 antibody (Neomarkers, cat#: MS-327-P: clone L87 and e2-4001 (Ab-10)), (LabVision Corp., Freemont, Calif.) eluted from the blot on the left.

The isolated functional antibody can then be used to screen a library of peptides to identify candidate antibody epitopes.

The isolated antibody is contacted with the library and positive beads are sequenced to identify the epitope. Positive beads can be visualized by using standard labeling techniques i.e., biotin, enzymes, fluorochromes, and similar reagents. Once the epitope sequence is determined, the sequence of this peptide can be compared to the specific list of candidate genes having the appropriate sized expression product to identify the gene expression product sequence that contains the epitope sequence identified in the peptide library screen.

Methods for Immunoprecipitating Polypeptide Antigens Direct Method

Standard immunoprecipitation techniques can be used when antibody of interest is abundant. For example, using the antibody of interest in well known Western blotting techniques as described hereinabove, a target sample/cell line that exhibits high level expression of the polypeptide antigen is initially identified. The target sample/cell line is then metabolically labeled with, for example ³⁵S-Methionine, and using standard techniques or the methods described herein. Target cells are gently broken open in solution. Serum antibody is then added to and incubated with the radioactive cell lysate (typically 1-2 hours at 4° C. with rotation) and collected on, for example, Protein A Sepharose beads. The antibody and antigen is bound to the solid support allowing washing and concentration of the sample. After removing the washing buffer (e.g., PBS/Tween-20), the beads are incubated with SDS-PAGE sample buffer, heated to 95° C. for 3 minutes and loaded onto a standard protein gel. When the gel is finished running, one of the glass plates is separated to expose the gel (still attached to one of the plates). A sheet of plastic wrap may be placed over the gel to prevent the gel from drying out. The gel is then placed on X-ray film for an appropriate period of time in order to detect the position of the labeled antigen. Upon detection of the position of the antigen, the plastic wrap is removed from the gel, and the gel is aligned precisely with the exposed X-ray film, and the portion of the gel corresponding to the position of the labeled antigen is excised uing a scalpel and recovered from the gel. The recovered antigen is analyzed by standard methods, for example MALDI-TOF MS.

Tracer or Indirect Method.

This method can be utilized when there is limiting amounts of antibody available. This method is similar to the Direct method except that the antibody of interest is eluted from the immunoblot and then used to immunoprecipitate its corresponding antigen from an ³⁵S-Methionine-labeled cell lysate. In the event that the amount of immunoprecipitated antigen recovered is insufficient to allow for sequencing, the labeled immunoprecipitated antigen will serve as a ‘tracer’ to identify and recover additional amounts of antigen. Accordingly, the labeled ‘tracer’ antigen is mixed in with a larger amount of cold (unlabeled) cell lysate of the same target sample/cell line used for the previous immunoprecipitation and the proteins are separated on a gel as described hereinabove. Detection of the labeled ‘tracer’ antigen not only identifies its own position on the gel, it additionally identifies the position of a much greater amount of corresponding unlabeled protein antigen that has co-migrated along with it on the gel. As described hereinabove, the gel is then exposed to film for an appropriate period of time in order to detect the position of the labeled ‘tracer’ antigen and corresponding co-migratory unlabeled protein antigen. Once excised from the gel using the method described herein. The antigen can be sequenced by standard methods or used to further isolate antigen or antibody.

Peptide Library Screening.

A combinatorial library comprised of random peptides is prepared with the 19 common amino acids (excluding cysteine) each having a fixed amino acid position at its carboxy-terminus which is bound to the solid phase support via a linker. The fixation of the carboxy-terminal position of the peptides provides for equal release of the peptides from the solid support. The library has a complexity of 19⁶ or approximately 47 million peptide species. In one embodiment, the library is comprised of random 6-mers. In alternative embodiments, the library may be comprised of partially degenerative peptides having one or more fixed or invariant positions, i.e., 7- to 10-mers.

In a typical antibody capture assay, the unlabeled antigen/epitope is immobilized on a solid support and the antibody is allowed to bind to the immobilized antigen/epitope, the antibody can be labeled directly or can be detected by using a labeled secondary reagent that will specifically recognize the antibody.

Since most immunochemical techniques have a certain background, and as the abundance of a particular antigen decreases relative to the other proteins, the ability of an antibody to distinguish the correct antigen from the background can be reduced. This is particularly true for polyclonal antibodies. Any technique that allows the antigen to be identified specifically among the background noise can be used as a secondary technique.

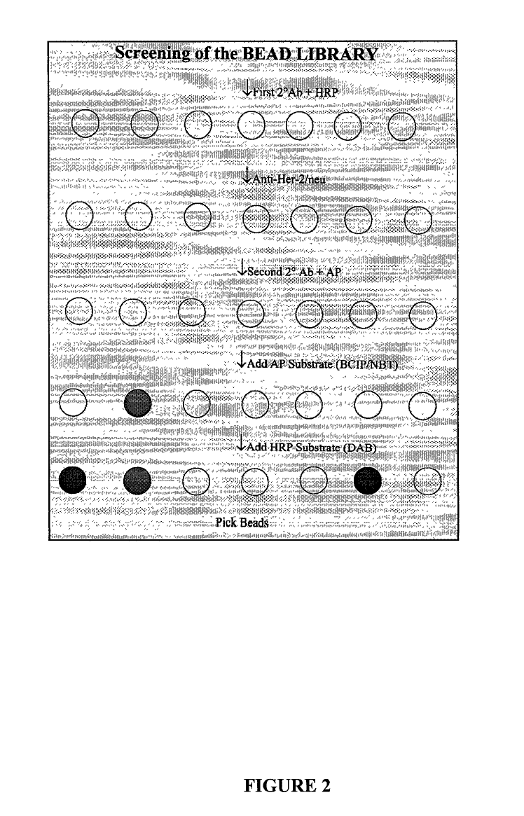

When utilizing an “off-bead” screening technology, enrichment of the antibody by the use of blocking antibodies is preferred. In one embodiment of the invention method (see FIG. 2) the antibodies utilized were designated as follows: (a) the 1° antibody is the antibody eluted off of the Western blot; (b) the “first” 2° antibody, specific for human IgG constant region, is the “blocking antibody” and is also enzyme labeled for use in an indirect detection assay; (c) the “second” 2° antibody is the “real or actual 2° antibody” and is identical to the “first” 2° or blocking antibody in every aspect, with the exception of its label. For example, a single animal was immunized with a single immunogen (i.e., human IgG CR), its sera collected and divided into two aliquots, one aliquot was derivatized into the “first” 2° (blocking) antibody having horseradish peroxidase (HRP) label, and the second aliquot was derivatized into the “second” 2° (real or actual) antibody having alkaline phosphatase (AP) label.

In one embodiment of the invention, the peptide library is screened as follows. The library is first saturated with the “first” 2° (blocking) antibody. After several washes to remove the blocking antibody, the 1° antibody is then contacted with the peptide library. The 1° antibody is removed from the library after several washing steps. Next, the “second” 2° (real or actual) antibody is contacted with the bead library. After washing the “second” 2° (real or actual) antibody off, 3′,3′-diaminobenzidine tetrahydrochloride (DAB), the substrate for the AP-labeled “second” 2° (real or actual) antibody is added to the library-staining beads with peptides specifically bound a purplish hue (shown as black in FIG. 2). After several washes to remove the DAB, the library was contacted with 5-bromo-4chloro-3-indolyl-phosphatate/4-nitro blue tetrazolium chloride (BCIP/NBT), the substrate for the HRP-labeled “first” 2° (blocking) antibody-staining beads with peptides specifically bound a brownish hue (shown as gray in FIG. 2). Since the “second” 2° antibody should only bind to the human constant region on the 1° antibody, any bead having a peptide to which the 1° antibody bound will stain purple. The “purple” bead(s) is/are readily detectable and distinguished from the remainder of the library beads which are a mixture of brown and clear beads. The positive bead(s) is/are then isolated and the peptide(s) is/are sequenced.

In another embodiment of the invention, library screening is accomplished “off bead” by arraying the beads containing the peptides in 96-well teflon filter bottom plates at, for example, 10,000 beads/well. A portion of peptide from every bead in each well is chemically released (as a peptide pool) and collected in a replica plate (standard 96-well plate). An aliquot of released peptide pool is then spotted onto, or alternatively a 96-well vacuum manifold can be used to deposit a fixed amount of released peptide onto, a membrane such as nitrocellulose. The eluted antibody of interest can then be used to perform a Western blot on the membrane containing the released peptides. Upon identification of a positive antibody:peptide complex on the Western blot, the beads contained in the well from which the ‘positive’ peptide pool was released can be re-arrayed into a new 96-well filter plate (at approximately 100 beads/well). As before, a portion of peptide from every bead in each well is released as a peptide pool, the pool then transferred to a solid membrane support and tested for reactivity with the antibody. At this point there should be a single positive well corresponding to the peptides released from the ˜100 bead pool. These ˜100 beads are re-arrayed into a new 96-well plate (now ˜1 bead/well), a portion of the solid-phase peptide released, and transferred to a membrane support. Probing the membrane once more with the antibody of interest will identify the well containing the single bead that contains the reactive peptide. Residual peptide on the positive bead can be directly sequenced by Edman degradation or other suitable methods known in the art.

Preparation of Gene Expression Database Using SAGE

SAGE (Serial Analysis of Gene Expression) is described in U.S. Pat. No. 5,695,937. SAGE is a technique for rapidly identifying and analyzing thousands of gene transcripts. Using SAGE, sequence tags corresponding to expressed genes can be identified and analyzed. A “tag” or “SAGE tag” is a short polynucleotide sequence, generally under or about 20 nucleotides that occur in a certain position in messenger RNA. The tag can be used to identify the corresponding transcript and gene from which it was transcribed. Application of a SAGE database is employed to identify the polypeptide of interest. First, the expression data for the target sample/cell line expressing the protein to which the antibody bound (i.e., the ‘positive’ target sample/cell) is examined to identify all genes over-expressed in said target sample/cell line. Genes so identified comprise a “preliminary list of candidate genes” (i.e., over-expressed in the positive target sample/cell). Second, the expression data for each gene in the “preliminary list” is examined in the target sample/cell lines that did not contain a protein (antigen) uniquely bound by an antibody present in the serum of a subject (i.e., the ‘negative’ target sample/cells). After determining the gene expression levels in the negative target samples/cells, genes not meeting the sorting criteria (described above) are eliminated and a refined “subset of candidate genes” is defined, e.g., genes that are over-expressed in the ‘positive’ target sample/cells and that are under-expressed in the ‘negative’ target sample/cells. Third, the candidate genes in the refined “subset” are further sorted to identify and select a gene that expresses a protein having a molecular weight approximately equal to the molecular weight observed of the antibody-bound protein (antigen) in the Western blot analysis of the subject's serum. Those of skill in the art appreciate that oftentimes the apparent molecular weight of a protein as determined from, for example, Western blot analyses, may present slightly larger than its actual molecular weight. Therefore, any gene in the refined “subset” that does not express a protein having the pre-selected molecular weight is eliminated, further narrowing the number of genes that meet the criteria for the “short list”, e.g. over-expressed in positive target sample/cells, under-expressed in negative target sample/cells, having a protein product of a specific pre-selected molecular weight.

SAGE can identify the antigen-encoding gene from the expression data and protein size analysis described above, e.g., where only a single candidate gene meets all criteria for the “short list”. However, there may also be instances where the “short list” consists of multiple candidate genes that meet the criteria. In such instances the invention further provides methods for identifying the target protein precisely. In order to perform such additional analyses, the antibody (that complexed with the polypeptide-antigen in the Western analysis) is further employed. In circumstances where the amount of available antibody is limited, for example, as may be the case when the antibody is from the serum sample of a human patient, in the methods of the present invention, the serum antibody is removed from the solid support in a manner such that the antibody's functional activity is retained. In one embodiment of the invention, the isolated antibody is used to immunoprecipitate the polypeptide antigen. In another embodiment, the isolated antibody is used to screen a library of peptides to identify candidate antibody epitopes. Positive candidate epitopes can be visualized by using standard labeling techniques (i.e., radioisotopes, enzymes, biotin, fluorochromes, and the like). The amino acid sequence of the candidate epitope (peptide) is then determined and compared with the amino acid sequences of the gene expression products encoded by genes in the pre-selected “short list”. Polypeptide sequences encoded by genes in the pre-selected “short list” that contain the amino acid sequence of the candidate epitope (peptide) will define the natural epitope recognized and bound by the antibody.

SAGE data can be sorted to identify differentially expressed genes that encode the cell surface targets recognized by antibodies shown to possess anti-tumor reactivity. Once the antigen is identified, the target protein antigen can be further assayed against serum samples obtained from human cancer subjects in order to determine whether the target protein antigen is naturally immunogenic.

Antigen Identification with MALDI-TOF

With knowledge of a nucleotide or gene candidate list and the apparent molecular weight of the antigen of interest, it may still not be possible to identify the legitimate antigen definitively. In cases where the solid-phase library screening does not yield useful information, a second technique, MALDI-TOF (shown in FIG. 5) may be coupled to the nucleotide sequence homology analysis in place of the library screen. This method allows generation of a list of candidate antigens by a method independent of genetic analysis.

Since there can be only one ‘correct’ antigen on any list of nucleotide sequences or databases and the same antigen must be present on the MALDI-TOF list, a comparison of the two can rapidly identify the antigen of interest. Furthermore, successful analysis can be obtained using antigen obtained from a standard 1-dimensional protein gel. The reason that there is little or no overlap between the ‘false positives’ on either list is due to the very different criteria that led to each set of false positives being present. That is, there appears to be no consistent causal relationship between a false positive that has a similar expression pattern to the correct antigen (SAGE) and a false positive that happens to co-migrate with the correct antigen or is erroneously identified by database searching (MALDI-TOF).

Epitope Mapping with MALDI-TOF

A purified or semi-purified antigen preparation and the antibody of interest are required reagents. First, the intact antigen is digested with an enzyme (e.g., trypsin, chymotrypsin) or chemical (e.g., CNBr) method in order to produce peptides of predictable molecular weights. Next, the antibody is added to the peptide mixture and allowed to “capture” the peptide that contains its linear recognition sequence. The antibody-peptide complexes can be collected on, for example, protein A-coated magnetic beads to allow for easy washing away of non-bound peptides. After washing, the antibody complexes can be denatured by adjusting the pH of the solution, releasing the bound peptides that contain the epitope of interest. By performing MALDI-TOF mass spectrometry on the eluted peptides, the molecular weight of the peptide fragment can be determined. This “actual” molecular weight can be compared to a list of theoretical “calculated” molecular weights to identify the sequence of the peptide fragment that contains the epitope.

Ambiguous results (using the above method) can occur in the following situations: (1) where ‘contaminating’ peptides co-purify with the legitimate epitope-containing peptide, in such case more than one molecular mass will be detected in the MS analysis; and (2) where the minimal essential epitope could be disrupted during the digestion of the antigen (i.e., if it harbors a cleavage consensus sequence), in which case either no masses or only contaminating masses will be identified by MS analysis.

In either situation (described above), the ambiguity can often be resolved as follows. Identical samples can be processed using different antigen digestion methods (preferably methods with mutually exclusive consensus sequences). This greatly enhances the chances of not destroying the minimal epitope in both digests. The masses retrieved from both digests are compared to identify the correct epitope-containing peptide. Comparison of the digest results may show that the epitope resides in an overlap region between peptides identified from each digest, further refining the position of the minimal essential epitope. In the event that one of the digestion methods did destroy the epitope, one could then deduce that the correct epitope-containing peptide in the other digest must contain a cleavage consensus sequence specific to the first digest.

### EXAMPLE NO. 1

### Screening a Combinatorial Peptide Library for αHer-2 Antibody-Reactive Peptides

Two novel epitopes (EYLGLDYQI; SEQ ID NO:1) and (EYLGLEMDV, SEQ ID NO: 2) that correlate with the HER-2 antigen (EYLGLDVPV, SEQ ID NO: 3), and the polynucleotides encoding them, were identified using the methods shown in FIGS. 2 and 3. Because the peptide library screen was performed “off-bead” it was necessary to obtain an enriched sample of antigen or polypeptide using the Indirect or Tracer Method described supra.

The antibodies used were, Anti-Her2/neu: Neomarkers cat# MS-599-P1: clone 3B5 (Ab-15) (LabVision Corp., Freemont, Calif.), goat anti-mouse IgG+HRP : Sigma cat# A9917 (pooled polyclonal serum from 2 goats), and goat anti-mouse IgG+AP: Sigma cat# A1293 (polyclonal serum from 1 goat: the serum from this particular goat is also part of the pooled polyclonal serum described immediately hereinabove).

10⁷ beads (dry weight in an orange top 50 ml tube=20.43 g {beads=7.2 g}) were blocked in 5% BLOTTO for >30min. The beads were incubated with the goat anti-mouse IgG+HRP (1:2500) for 1.5 hrs while rotating at room temperature (“RT”). The beads were washed several times with PBS+0.1% Tween20 and incubated with the anti-Her2/neu antibody Ab-15 (previously eluted from an immunoblot) in 20 ml for 2 hrs at RT. The beads were washed several times with PBS+0.1% Tween20. The beads were incubated with the goat anti-mouse IgG+AP (1:2500) for 1.5 hrs while rotating at RT.

The beads were washed O/N @ 4° C. while rotating. The substrate for AP (alkaline phosphatase) was added, BCIP/NBT (5-bromo-4-chloro-3-indolyl phosphate/4-nitro blue tetrazolium chloride), (Zymed cat# 00-2210; So. San Francisco, Calif.) and incubated with the beads for 30 min at RT. The beads were washed several times with PBS. The substrate for HRP (horseradish peroxidase) was added, DAB (3,3′-diaminobenzidine tetrahydrochloride), (Vector labs cat# SK-4100; Burlingame, Calif.) and incubated for 10 min. The beads were washed several times with PBS. The beads were viewed under a dissecting microscope. The violet-stained beads were selected and arrayed in a filter bottom 96-well microtiter plate. Peptide was released from 2 of the arrayed beads by chemical cleavage with trifluoroacetic acid (TFA) and then subsequently sequenced.

FIG. 3 shows the Western blot from which the Her-2 antibody was eluted (top left) and one of the “positive” beads that was sequenced (bottom left). The figure on the bottom right shows an alignment of the two library sequences with a region of the Her-2 protein. These sequences are compared to the candidate gene database list. This region lies within the 15 amino acid peptide that was originally used to generate the αHer2 antibody.

### EXAMPLE NO. 2

### Antigen Identification Using MALDI-TOF

In this example, unfractionated longitudinal serum samples from a melanoma patient treated with a MART-1 vaccine delivered in an adenoviral vector and that had a complete clinical response were analyzed.

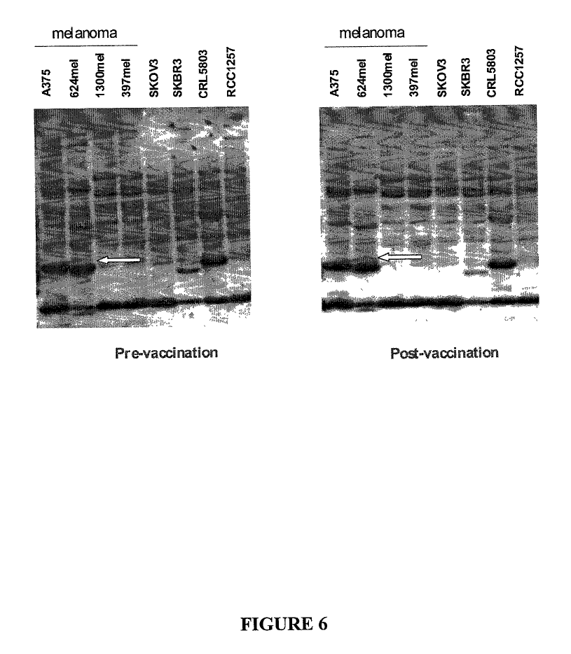

An IgM antibody activity that correlated with multisite regression of metastases was identified. The cell lines used to prepare the lysates that the serum was blotted against were all tumor cell lines for which SAGE data was available. See FIG. 6.

Having identified a band of interest, an identical protein gel used for the analysis shown above was prepared using the third longitudinal serum sample. This band was cut out of the gel and digested with trypsin. MALDI-TOF MS was performed on the tryptic peptides and 99 molecular masses were elucidated. These masses were analyzed for matches with calculated tryptic digests of all proteins in the SwissProt database using the program MS-FIT developed by Peter Baker and Karl Clauser at UCSF (see http://prospector.ucsf.edu/ucsfhtml3.4/msfit.htm).

The search parameters are summarized below:

$$\begin{matrix}\begin{matrix}Database \\ Statistics\end{matrix} \{ \begin{matrix}Database \text{:} SwissProt . 111000 \\ Mol . Wt . Range \text{:} 1 , 000 –50 , 000 Da ( selects 66 , 218 entries ) \\ PI Range \text{:} Full ( 90 , 195 entries ) \\ Species search \text{:} Human ( 6 , 187 entries ) \\ [ Combined MW , PI , Species selects 3 , 801 entries ]\end{matrix} \\ Sample Statistics \{ \begin{matrix}Min \# peptides to match \text{:} 4 \\ Digest \text{:} Trypsin \\ Max . \# missed cleavages \text{:} 1 \\ \# Input Masses \text{:} 99\end{matrix} \\ Results \{ {{MS \text{-} Fit search selects 187 entries ( Top 100 collected ) }_{}}^{}\end{matrix}$$

Having generated a MALDI-TOF antigen candidate list, with the protein shown above having the highest score, the top 100 on this list were checked for expression patterns in the cell lines using the a database library, in this case a SAGE library generated from melanoma cell lines. Only protein of roughly the correct molecular weight that roughly matched the Western blot results.

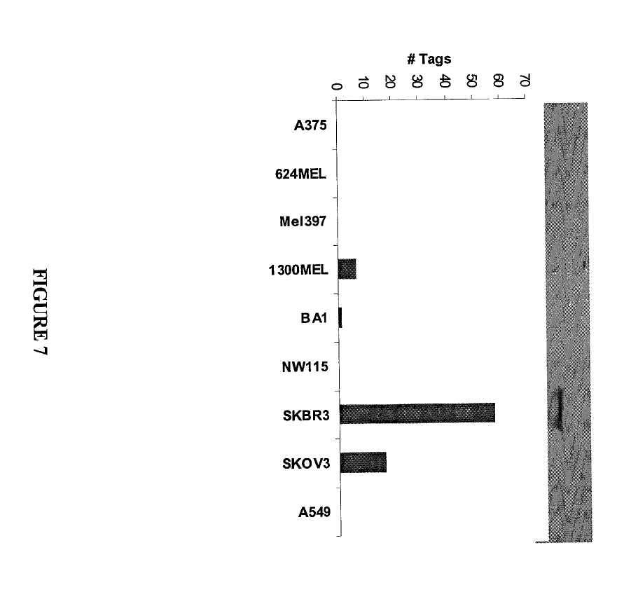

In order to confirm the antibody, a commercially available monoclonal antibody was used to immunoprecipitate the protein from a whole cell lysate of a tumor cell line that was known to express this protein. The whole cell lysate, the immunoprecipitated protein, and the supernatant of the immunoprecipitated sample were analyzed by western blotting with the third longitudinal patient serum sample. That blot is shown in FIG. 7.

### EXAMPLE NO.3

### Identification or Confirmation of Antigens Using Animal Models

Immunocompetent mice are immunized with whole tumor cells using the method of Fradet et al. (1984) P.N.A.S. USA 81: 224, tumor cell membranes or serum-free conditioned medium from cultured tumor cells. Total serum is collected from sacrificed mice and total antibodies are collected using the method of Reilly et al. (2001) Cancer Research 61:880. The mouse antibodies are absorbed to normal cells (fibroblasts, lymphocytes) to eliminate pan reactive mouse antibodies recognizing ubiquitous antigens. In vitro or in vivo assays can be conducted to assess the functional properties of the enriched antibody preparation. For instance enriched antibodies can be added to human tumor cells in the presence of complement and peripheral blood mononuclear cells (PBMCs) in vitro to determine if the antibodies can mediate antibody dependent cell-mediated cytotoxicity (ADCC) by the method of Scott et al. (2000) Cancer Research 60:3254. Alternatively enriched antibodies can be adoptively transferred to immunodeficient mice by the method of Reilly et al. (2001) supra, of a similar genetic background to the immunocompetent mice used to generate the antibodies to determine if they confer protection from challenge by the human tumor cells. If the antibody preparation are shown to kill human tumor cells in vitro or retard human tumor cell growth in vivo, a biologically active anti-human cancer cell antibody must be present. Western blot analyses using the biologically active antibody are tested for reactivity against human cancer cells that are affected by the antibody relative to other human cells (cancerous and normal) that are not to rapidly identify the cognate antigens for therapeutic antibodies. Autologous or allogeneic human cancer cells or mouse cancer cells or their respective membrane fractions or shed antigens are useful to generate biologically active polyclonal antibodies in immunocompetent mice. However, the probability of generating biologically active antibodies will be greater if the human tumor cells used to generate antibodies in immunocompetent mice are the same as those used to challenge immunodeficient mice.

### EXAMPLE NO. 4

### Use of Methods to Identify Antigens Implicated in an Autoimmune Disease, Systemic Lupus Erythematosus

A number of different cytokines have been implicated in the development of systemic lupus erythematosus (“SLE”). Interleukin 10 for instance appears to be elevated in SLE patients suggesting that it may play a role in the development or progression of the autoimmune disease Llorente et al. (1955) Journal of Experimental Medicine 181:839) administered anti-IL-10 antibodies to immunodeficient mice that had previously been injected with peripheral blood mononuclear cells from SLE patients and observed that this treatment reduced the concentration of human autoantibodies in the serum of treated mice. However other secreted factors (interleukin 1) and cell surface markers (CD40L) have been reported to be elevated in SLE patients and as the relative contributions of these molecules to the pathogenesis of SLE are unknown, the arbitrary selection of any one target (IL-10) for neutralization may fail in the clinic.

As an alternative to generating an antibody to one defined target, immunocompromised mice are immunized with SLE patient peripheral blood mononuclear cells (“PBMCs”), or isolated cells from SLE patients (e.g., dendritic cells, T cells or B cells) or cell membranes or serum-free conditioned medium derived from such cells. Total serum is collected from sacrificed mice and total antibodies are collected. The mouse antibodies are absorbed to normal human cells (fibroblasts, lymphocytes) to eliminate pan reactive mouse antibodies recognizing ubiquitous antigens. In vitro or in vivo assays are conducted to assess the functional properties of the enriched antibody preparation. For instance enriched antibodies are added to SLE patient cells in the presence of complement and PBMCs in vitro to determine if the antibodies can mediate ADCC. Alternatively enriched antibodies are adoptively transferred to immunodeficient mice (of a similar genetic background to the immunocompetent mice used to generate the antibodies) to determine if they modulate the behavior of SLE patient PBMCs that have been injected into the immunodeficient mice. If the antibody preparation is shown to modulate SLE patient PBMCs in vitro or in vivo, a biologically active antibody recognizing a SLE target must be present. One then conducts Western blot analyses using the biologically active antibody and tests for reactivity against patient SLE cells that are affected by the antibody relative to other human cells (normal counterparts from healthy donors) that are not, to rapidly identify the cognate antigens for therapeutic antibodies.

### EXAMPLE NO. 5

### Identification of Antigens in Conditions where Inappropriate Expression of Protein is Associated with Disease State, Polycystic Kidney Disease

Immunocompetent mice are immunized with polycystic kidney cells or cell membranes or serum-free conditioned medium derived from such cells. Total serum is collected from sacrificed mice and total antibodies are collected. The mouse antibodies are absorbed to normal cells (fibroblasts, lymphocytes, normal kidney) to eliminate pan reactive mouse antibodies recognizing ubiquitous antigens. In vitro or in vivo assays are conducted to assess the functional properties of the enriched antibody preparation. For instance enriched antibodies are added to polycystic kidney cells in the presence of complement and PBMCs in vitro to determine if the antibodies can mediate ADCC. Alternatively enriched antibodies are adoptively transferred to mice that spontaneously develop polycystic kidney disease (of a similar genetic background to the immunocompetent mice used to generate the antibodies) to determine if they inhibit the development of cysts in vivo. If the antibody preparation is shown to be cytostatic or cytotoxic for cysts, an antibody of potential therapeutic utility must be present. Western blot analyses is conducted using the biologically active antibody and tests for reactivity against cystic cells that are affected by the antibody relative to other cells that are not to rapidly identify the cognate antigens for therapeutic antibodies. Either human or non-human polycystic kidney cells are used to generate the polyclonal antibodies and prepare lysates for Western blot analysis and subsequent antigen identification.

It is to be understood that while the invention has been described in conjunction with the above embodiments, that the foregoing description and examples are intended to illustrate and not to limit the scope of the invention. Other aspects, advantages and modifications within the scope of the invention will be apparent to those skilled in the art to which the invention pertains.

What is claimed is:

1. A method to identify the polypeptide correlating with a phenotype of interest, wherein the polypeptide specifically recognizes and binds a serum antibody, said method comprising identifying the polypeptide common to a list of characterized genes, wherein said genes are differentially expressed in one or more relevant cells or tissues and a list of characterized polypeptides, thereby identify said polypeptide correlating with said phenotype of interest.

2. The method of claim 1, wherein the genes of the list are characterized by properties of the gene product, wherein said properties are selected from the group consisting of specific reactivity with the serum antibody, highly expressed in the relevant cell line or tissue, little or no detectable expression in the relevant cell line or tissue, and uniquely expressed in the relevant cell line or tissue.

3. The method of claim 2, wherein two or more properties characterize the properties of the gene product.

4. The method of claim 2, further comprising the property of molecular weight.

5. The method of claim 3, further comprising the property of molecular weight.

6. The method of claim 1, wherein the list of characteristics of the proteins of the list is a selected from mass, reactivity with the serum antibody, peptidase digestion pattern, enzymatic digestion pattern and MOLDI-TOF selection criteria.

7. The method of claim 6, wherein two or more properties characterize the properties of the gene product.

8. The method of claim 6, further comprising the property of molecular weight.

9. The method of claim 7, further comprising the property of molecular weight.

10. The method of claim 6, wherein MOLDI-TOF selection criteria are selected from the group consisting database selection, species, type of digest, CNBr, number of miscleavages, molecular weight range, contamination indication, and mass accuracy.
